(19) United States
(12) Patent Application Publication
(10) Pub. No.: US 2010/0129317 A1
(43) Pub. Date: May 27, 2010

(54) AZOLE NUCLEOSIDES AND USE AS INHIBITORS OF RNA AND DNA VIRAL POLYMERASES

(75) Inventors: Jeffrey B. Arterburn, Las Cruces, NM (US); Colleen B. Jonsson, Birmingham, AL (US); William B. Parker, Birmingham, AL (US)

Correspondence Address: CONNOLLY BOVE LODGE & HUTZ LLP, 1875 EYE STREET, N.W., SUITE 1100, WASHINGTON, DC, 20006, US

(73) Assignee: SOUTHERN RESEARCH INSTITUTE, Birmingham, AL (US)

(21) Appl. No.: 12/440,697

(22) PCT Filed: Sep. 11, 2007
(86) PCT No.: PCT/US2007/078139

§ 371 Date: Jan. 22, 2010

Related U.S. Application Data
(60) Provisional application No. 60/843,398, filed on Sep. 11, 2006.

(51) Int. Cl. A61K 38/20; A61K 38/21; A61K 31/7034; A61K 31/7056; A61K 31/7076; C07H 19/167; C07H 19/056; C07H 19/052; A61P 31/12

(52) U.S. Cl. 424/85.2; 424/85.4; 424/85.7; 514/26; 514/43; 514/45; 536/27.8; 536/28.7; 536/28.8

(57) ABSTRACT

Azole nucleosides represented by the formulae (I) and (II); wherein A=C or N B═C or N X═H; C₁-C₆ alkyl, cycloalkyl, alkenyl, cycloalkenyl, alkynyl, aryl, heterocyclo, halogen such as F, Cl, Br and I; OH, NH₂, NH—(C₁-C₆ alkyl, cycloalkyl, aryl or heterocyclo); Z═H; C₁-C₆ alkyl, cycloalkyl, alkynyl, aryl, heterocyclo, halogen such as F, Cl, Br, I; OH NH₂, NH—(C₁-C₆ alkyl, cycloalkyl, aryl or heterocyclo; E=(CH₂)HONHR; n is an interger from 0-6 and more typically 0-3; R¹⁼ aryl or heterocyclo; each of W, Y, R is individually selected from the group consisting of H; C₁-C₆ alkyl, cycloalkyl, alkenyl, cycloalkenyl, alkynyl, aryl, heterocyclo, halogen such as F, Cl, Br, and I; O, OH, Oalkyl, Oaryl, NH₂, NH(C₁-C₆ alkyl, cycloalkyl, aryl or heterocyclo); provided that at least one of W, Y, and R is other than H and wherein both W and Y together can be ═O; and each D individually is OH, Oalkyl, Oaryl, FL and H; pharmaceutically acceptable salts thereof, prodrugs thereof and mixtures thereof are provided. Compounds of this disclosure are useful as inhibitors of viral RNA and DNA polymerases such as, but not limited to, Influenza, hantaan Virus, Crimean Congo hemorrhagic fever virus, hepatitis B, hepatitis C, Polio, Coxsackie A and B, Rhino, Echo, orthopoxvirus (small pox), HIV, Ebola, and West Nile virus polymerases; and especially orthopoxvirus, HIV, and hepatitis B.

### TECHNICAL FIELD

The present disclosure relates to azole and especially diazines such as pyrazole and imidazole; triazine and purine compounds that are useful as inhibitors of viral RNA and DNA polymerases such as, but not limited to, influenza, Hantaan Virus (HTNV), Crimean Congo hemorrhagic fever virus (CCHF), Rift Valley Fever virus (RVFV), hepatitis B, hepatitis C, Polio, Coxsackie A and B, Rhino, Echo, orthopoxvirus (small pox), HIV, Ebola, and West Nile virus polymerases; and especially influenza, and Bunyaviridae family viruses such as Hantaan Virus, Crimean Congo hemorrhagic fever virus and Rift Valley Fever virus.

The present disclosure also relates to pharmaceutical compositions comprising the above disclosed compounds, as well as methods of using the compounds in inhibiting viral RNA and DNA polymerases and treating patients suffering from diseases caused by various RNA and DNA viruses and various cancers.

The present disclosure also relates to a method for producing the compounds of the present disclosure.

### BACKGROUND

Viral diseases are one of the major causes of deaths and economic losses in the world. Out of various viral diseases, Influenza, HIV, HBV and HCV infections are more important and responsible for a large number of deaths. There are some drugs for HIV, only a few for HBV but no good drug for HCV. Hepatitis C is a viral liver disease, caused by infection with the hepatitis C virus (HCV). There are approximately 170 million people worldwide with chronic HCV infection, of which about 2.7 million are in the United States. HCV is a leading cause of cirrhosis, a common cause of hepatocellular carcinoma, and is the leading cause of liver transplantation in the United States. Currently, α-interferon monotherapy and α-interferon-ribavirin combination therapy are the only approved treatments for HCV.

It would be desirable to develop inhibitors of RNA and DNA viral polymerases.

### SUMMARY OF DISCLOSURE

In particular, the present disclosure relates to compounds represented by the formulae:

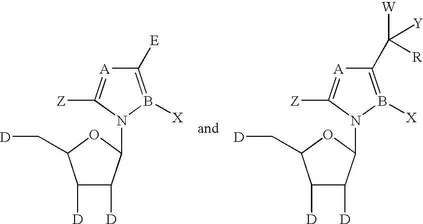

wherein A=C or N
B═C or N
X═H; C₁-C₆ alkyl, cycloalkyl, alkenyl, cycloalkenyl, alkynyl, aryl, heterocyclo, halogen such as F, Cl, Br and I; OH, NH₂, NH—(C₁-C₆ alkyl, cycloalkyl, aryl, or heterocyclo);
Z═H; C₁-C₆ alkyl, cycloalkyl, alkenyl, cycloalkenyl, alkynyl, aryl, heterocyclo, halogen such as F, Cl, Br, I; OH, NH₂, NH—(C₁-C₆ alkyl, cycloalkyl, aryl, or heterocyclo;
E=(CH₂)ₙONHR¹; n is an integer from 0-6 and more typically 0-3;
R¹=aryl or heterocyclo;
each of W, Y, R is individually selected from the group consisting of H; C₁-C₆ alkyl, cycloalkyl, alkenyl, cycloalkenyl, alkynyl, aryl, heterocyclo; halogen such as F, Cl, Br, and I; O, OH, Oalkyl, Oaryl, NH₂, NH—(C₁-C₆ alkyl, cycloalkyl, aryl, or heterocyclo);
provided that at least one of W, Y, and R is other than H and NH₂ and wherein both W and Y together can be ═O; and
each D individually is OH, Oalkyl, Oaryl, Fl and H;
pharmaceutically acceptable salt thereof, a prodrug thereof and mixtures thereof.

Another aspect of the present disclosure relates to pharmaceutical composition containing at least one of the above-disclosed compounds.

A further aspect of the present disclosure relates to a method for inhibiting RNA viral polymerase in a patient by administering to the patient at least one of the above disclosed compounds in an amount effective for inhibiting RNA viral polymerase.

A still further aspect of the present disclosure relates to a method for treating a patient suffering from an RNA viral infection which comprises administering to said patient an effective amount of at least one of the above disclosed compounds.

Still other objects and advantages of the present disclosure will become readily apparent by those skilled in the art from the following detailed description, wherein it is shown and described preferred embodiments, simply by way of illustration of the best mode contemplated. As will be realized the disclosure is capable of other and different embodiments, and its several details are capable of modifications in various obvious respects, without departing from the disclosure. Accordingly, the description is to be regarded as illustrative in nature and not as restrictive.

### SUMMARY OF DRAWINGS

FIG. 1 is a graph that illustrates that TA-18 is a subs ate for human adenosine kinase.

FIG. 2 is graph that illustrates that TA-18 was converted to phosphorylated metabolites in human CEM cells.

FIG. 3 shows graphs that illustrate that the treatment with TA-18 resulted in a decline in GTP levels.

FIG. 4 is a graph that illustrates the inhibition of adenosine kinase activity with iodotubercidin inhibited the metabolism of TA-18 in human cells.

FIG. 5 is a graph that illustrates that the inhibition of adenosine kinase activity with iodotubercidin also prevented the decline in GTP levels caused by TA-18.

FIG. 6 is a graph that illustrates that much less intracellular metabolites are formed from TA-18 than from ribavirin.

FIG. 7 is a graph that illustrates that treatment with ribavirin also caused a decrease in GTP levels in human cells.

### BEST AND VARIOUS MODES

In particular, the present disclosure relates to compounds represented by the following formulae:

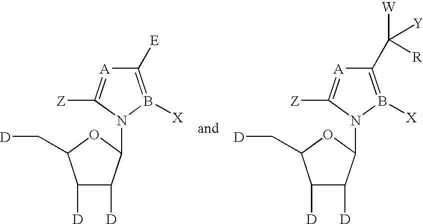

wherein A=C or N
B═C or N
X═H; C₁-C₆ alkyl, cycloalkyl, alkenyl, cycloalkenyl, alkynyl, aryl, heterocyclo; halogen such as F, Cl, Br and I; OH, NH₂, NH—(C₁-C₆ alkyl, cycloalkyl, aryl, or heterocyclo)
Z═H; C₁-C₆ alkyl, cycloalkyl, alkenyl, cycloalkenyl, alkynyl, aryl, heterocyclo, halogen such as F, Cl, Br, I; OH, NH₂, NH—(C₁-C₆ alkyl, cycloalkyl, aryl, or heterocyclo);
E=(CH₂)ₙONHR¹; n is an integer from 0-6 and more typically 0-3;
R¹=aryl or heterocyclo;
each of W, Y, R is individually selected from the group consisting of H; C₁-C₆ alkyl, cycloalkyl, alkenyl, cycloalkenyl, alkynyl, aryl, heterocyclo, halogen such as F, Cl, Br, and I; O, OH, Oalkyl, Oaryl, NH₂, NH—(C₁-C₆ alkyl, cycloalkyl, aryl, or heterocyclo;
provided that at least one of W, Y, and R is other than H and NH₂ and wherein both W and Y together can be ═O; and
each D individually is OH, Oalkyl, Oaryl, Fl and H;
a pharmaceutically acceptable salt thereof, a prodrug thereof and mixtures thereof.

The stereochemistry of the substituents in these compounds may be either (R) or (S) at the substituted positions. Of course mixtures of the different stereoisomers are contemplated.

Listed below are definitions of various terms used to describe this invention. These definitions apply to the terms as they are used throughout this specification, unless otherwise limited in specific instances, either individually or as part of a larger group.

The term “alkyl” refers to straight or branched chain unsubstituted hydrocarbon groups containing typically 1 to 6 carbon atoms, and more typically 1 to 3 carbon atoms.

Examples of suitable alkyl groups include methyl, ethyl and propyl. Examples of branched alkyl groups include isopropyl and t-butyl. Examples of suitable alkoxy groups are methoxy, ethoxy and propoxy.

The cycloalkyl groups typically contain 3-6 carbon atoms and include cyclopropyl, cyclobutyl, cyclopentyl and cyclohexyl.

Examples of halo groups are Cl, F, Br and I.

The alkenyl groups typically contain 2-6 carbon atoms and include ethenyl, propenyl and butenyl.

The cycloalkenyl groups typically contain 3-6 carbon atoms and include cyclopropenyl, cyclobutenyl, cyclopentenyl and cyclohexenyl.

The alkynyl groups typically contain 2-6 carbon atoms and include acetylenyl and propynyl.

The term “aryl” refers to monocyclic or multiring aromatic hydrocarbon groups typically containing 6 to 14 carbon atoms in the ring portion, such as phenyl, 2-naphthyl, 1-naphthyl, 4-biphenyl, 3-biphenyl, 2-biphenyl, and diphenyl groups, each of which may be substituted.

The term “heterocyclo” refers to saturated or unsaturated, single or multiringed groups.

Examples of multiring aromatic (unsaturated) heterocycle groups are 2-quinolinyl, 3-quinolinyl, 5-quinolinyl, 6-quinolinyl, 7-quinolinyl, 1-isoquinolinyl, 3-isoquinolinyl, 6-isoquinolinyl, 7-isoquinolinyl, 3-cinnolyl, 6-cinnolyl, 7-cinnolyl, 2-quinazolinyl, 4-quinazolinyl, 6-quinazolinyl, 7-quinazolinyl, 2-quinoxalinyl, 5-quinoxalinyl, 6-quinoxalinyl, 1-phthalaonyl, 6-phthalazinyl, 1-5-naphthyridin-2-yl, 1,5-naphthyridin-3-yl, 1,6-naphthyridin-3-yl, 1,6-naphthyridin-7-yl, 1,7-naphthyridin-3-yl, 1,7-naphthyridin-6-yl, 1,8-naphthyrdin-3-yl, 2,6-naphthyridin-6-yl, 2,7-naphthyridin-3-yl, indolyl, 1H-indazolyl, purinyl and pteridinyl.

Examples of single ring heterocycle groups are pyrrolyl, pyranyl, oxazolyl, thiazoyl, thiophenyl, furanyl, imidazolyl, pyrazolyl, pyridinyl, pyrazinyl, pyrimidinyl, 4-pyrimidinyl, 3-pyrimidinyl and 2-pyrimidinyl, pyridazinyl, isothiazolyl and isoxazolyl.

Examples of saturated heterocycle groups are pyrrolidinyl, imidazolidinyl, pyrazolidinyl, piperidinyl, piperazinyl, and morpholinyl.

The heterocycle groups contain N, O and/or S and typically contain 5 to 10 atoms in the ring(s), and typically contain 1, 2 or 3 heteroatoms (e.g. —N, O and S) in the ring.

If desired the above alkyl, cycloalkyl, alkenyl, cycloalkenyl, alkynyl, aryl and heterocyclo groups can be substituted. When substituted, such groups are typically substituted with halogen and/or alkyl substituents and/or (CH₂)ₙONH₂ wherein n is an integer from 0-6 and more typically 0-3. It is of course understood that the compounds of the present disclosure relate to all optical isomers and stereo-isomers at the various possible atoms of the molecule.

The compounds according to this disclosure may form prodrugs at hydroxyl or amino functionalities using alkoxy, amino acids, etc. groups as the prodrug forming moieties. For instance, the hydroxymethyl position may form mono-, di- or triphosphates and again these phosphates can form prodrugs. The hydroxy and hydroxymethyl groups may be converted to —OCH₂P(O)(OH)₂ and the prodrugs of phosphonates. The oxygen atom of the hydroxymethyl may be converted to CH₂ and then to CH₂P(O)(OH)₂ and the prodrugs.

Prodrug forms of the compounds bearing various nitrogen functions (amino, hydroxyamino, amide, etc.) may include the following types of derivatives where each R group individually may be hydrogen, substituted or unsubstituted alkyl, aryl, alkenyl, alkynyl, heterocycle, alkylaryl, aralkyl, aralkenyl, aralkynyl, cycloalkyl or cycloalkenyl groups as defined earlier.

(a) Carboxamides, —NHC(O)R

(b) Carbamates, —NHC(O)OR

(c) (Acyloxy)alkyl Carbamates, NHC(O)OROC(O)R

(d) Enamines, —NHCR(═CHCO₂R) or —NHCR(═CHCONR₂)

(e) Schiff Bases, —N═CR₂

(f) Mannich Bases (from carboximide compounds), RCONHCH₂NR₂

Preparations of such prodrug derivatives are discussed in various literature sources (examples are: Alexander et al. J. Med. Chem. 1988, 31, 318; Aligas-Martin et al., PCT WO pp/41531, p. 30). The nitrogen function converted in preparing these derivatives is one (or more) of the nitrogen atoms of a compound of the disclosure.

Prodrug forms of carboxyl-bearing compounds of the disclosure include esters (—CO₂R) where the R group corresponds to any alcohol whose release in the body through enzymatic or hydrolytic processes would be at pharmaceutically acceptable levels. Another prodrug derived from a carboxylic acid form of the disclosure may be a quaternary salt type

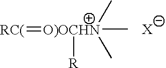

of structure described by Bodor et al. J. Med. Chem. 1980, 23 469.

Pharmaceutically acceptable salts of the compounds of the present disclosure include those derived from pharmaceutically acceptable inorganic or organic acids. Examples of suitable acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include alkali such as sodium and ammonia.

Some compounds within the scope of this disclosure are represented by the following:

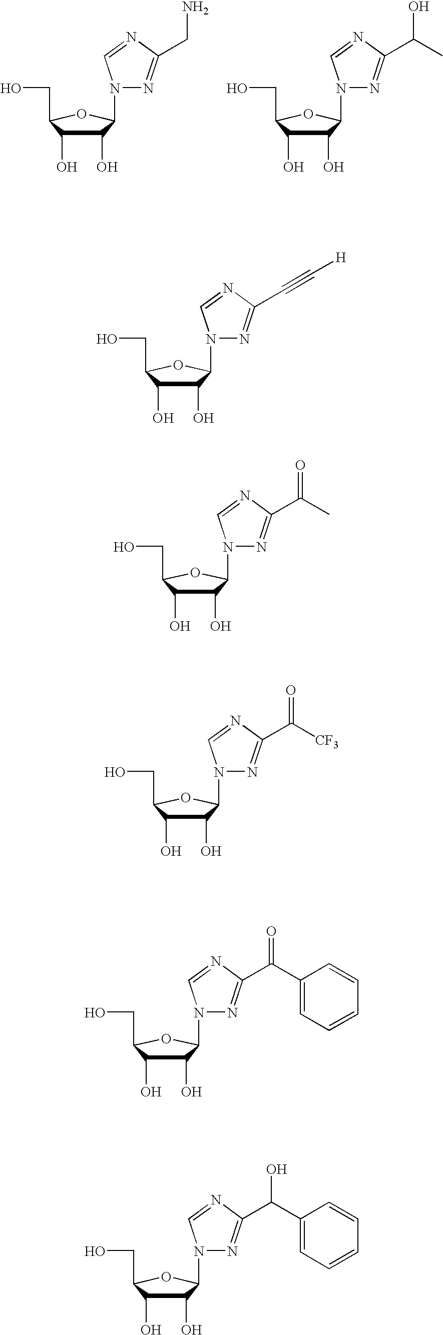

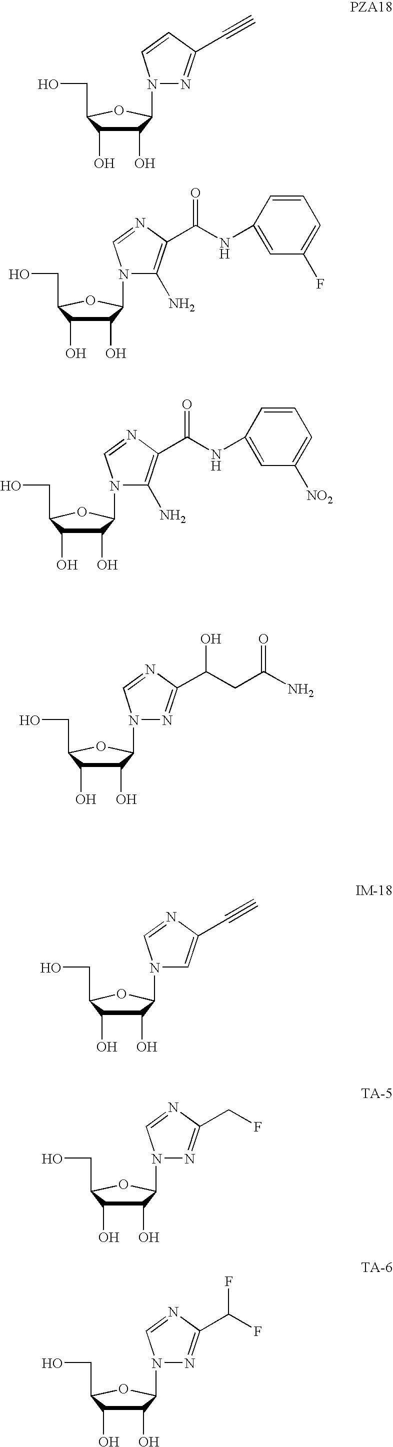

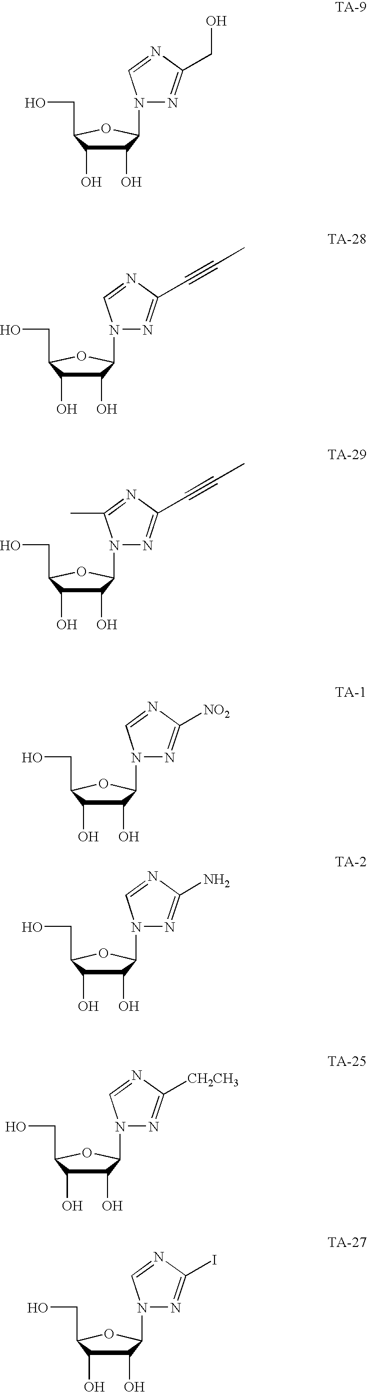

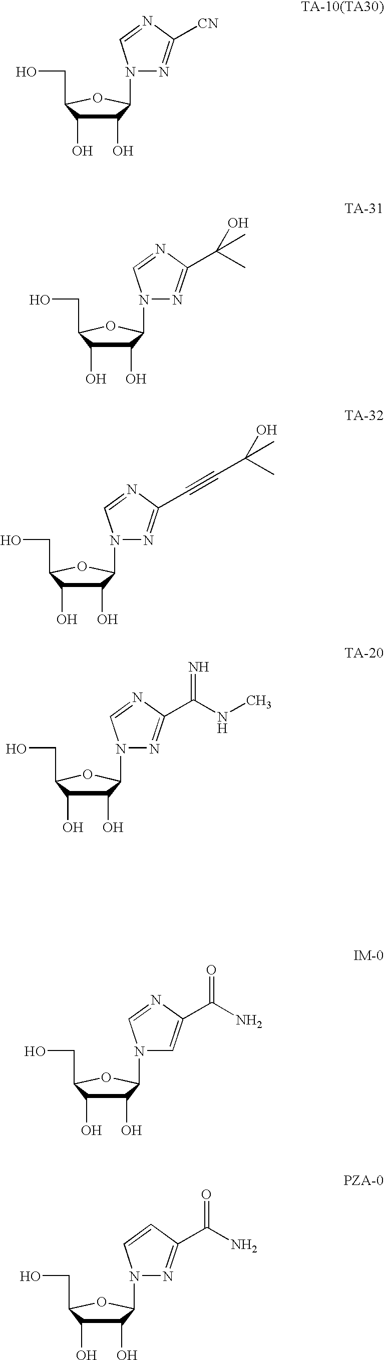

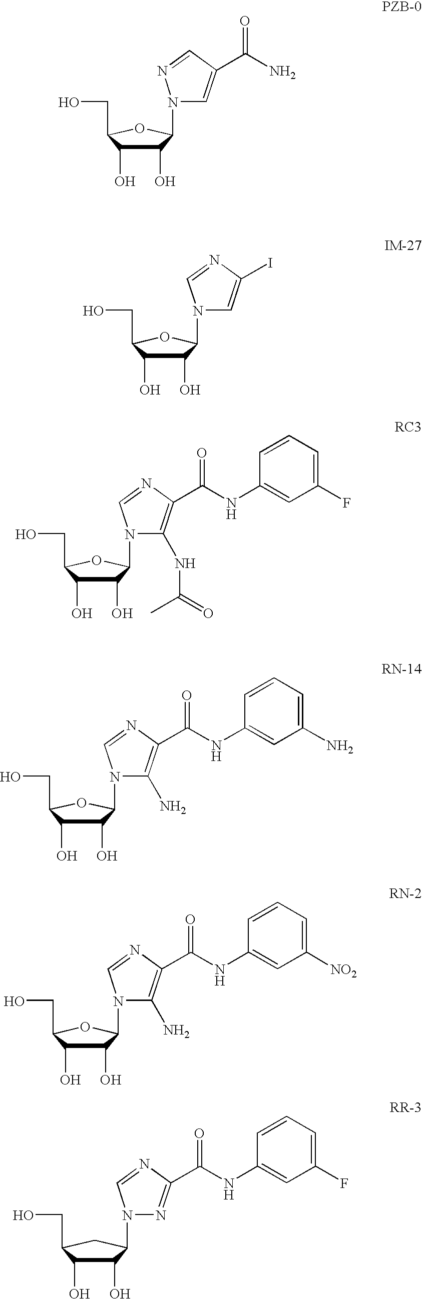

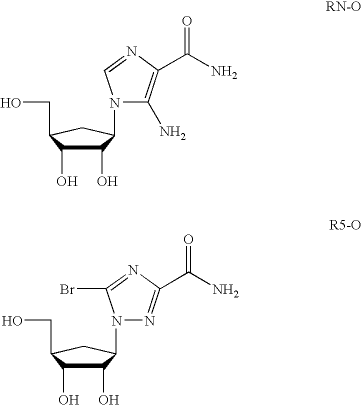

A representative example of a N-arylcarboxamide azole riboside is as follows:

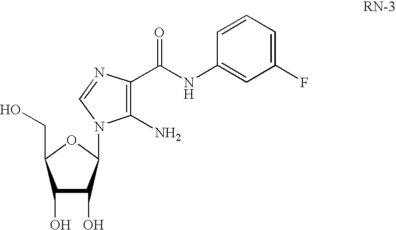

Representative examples of carbon-substituted azole ribosides according to this disclosure are as follows:

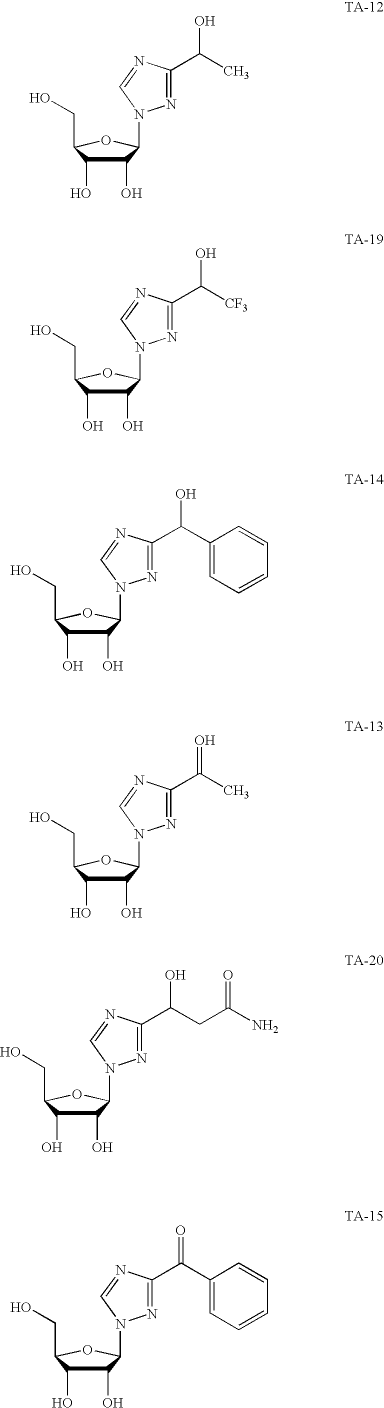

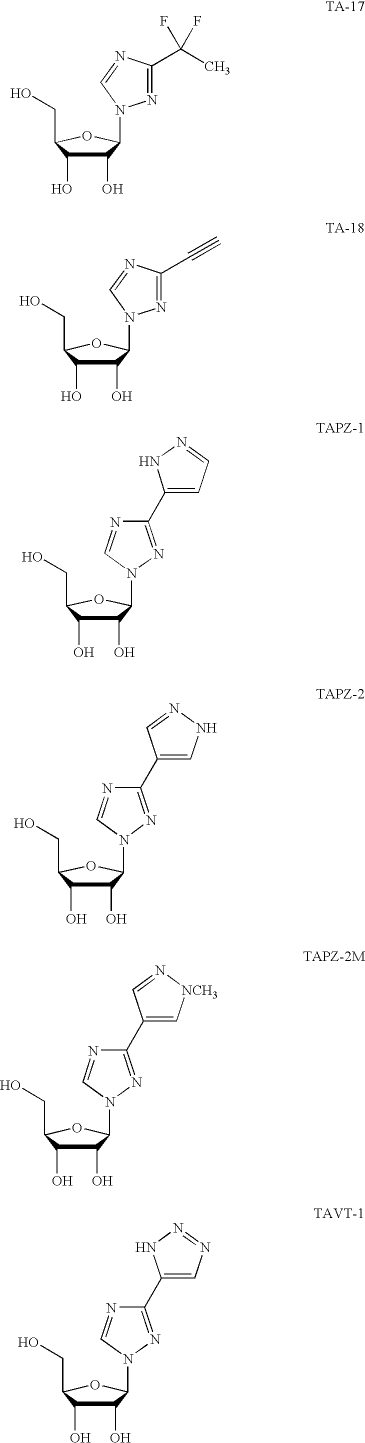

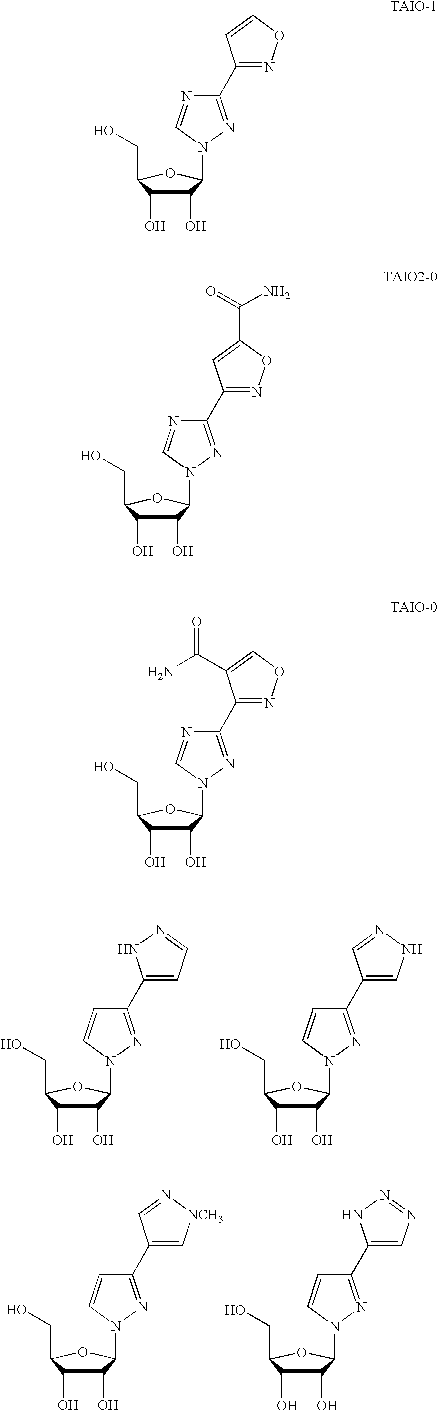

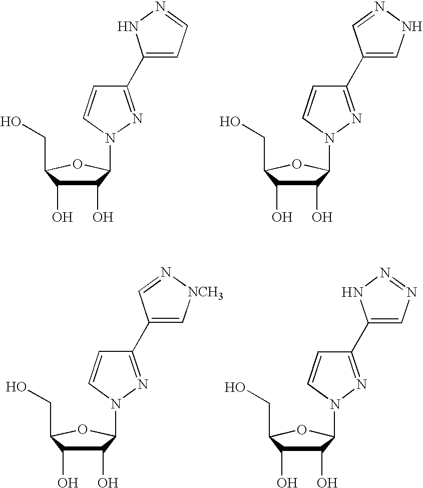

Structures of representative novel 1-β-D-ribofuranosyl-compounds that have been synthesized for antiviral screening are illustrated below:

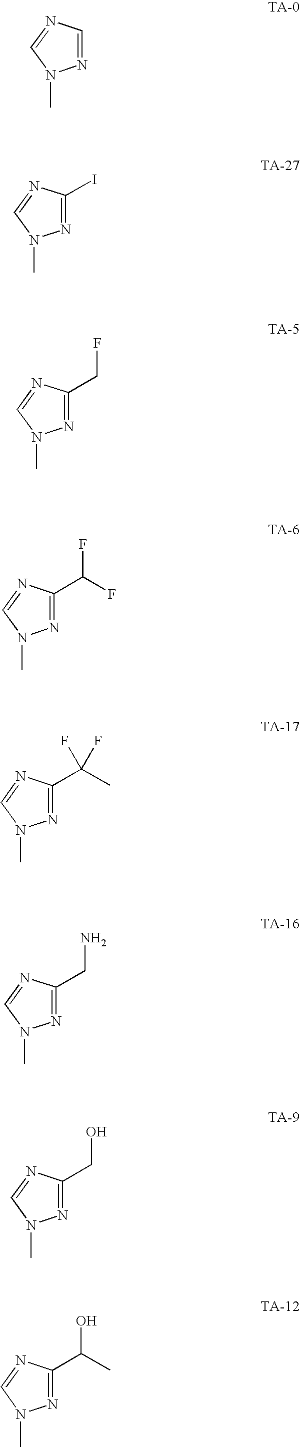

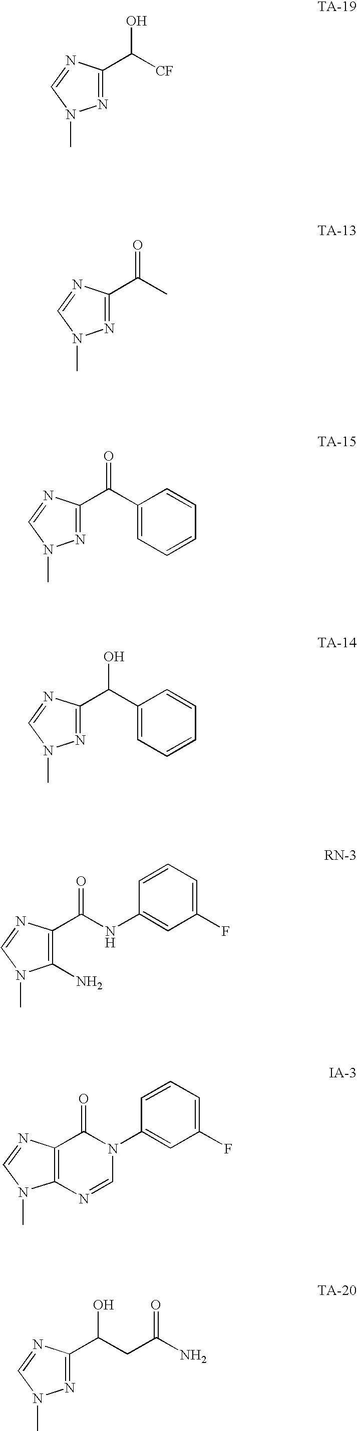

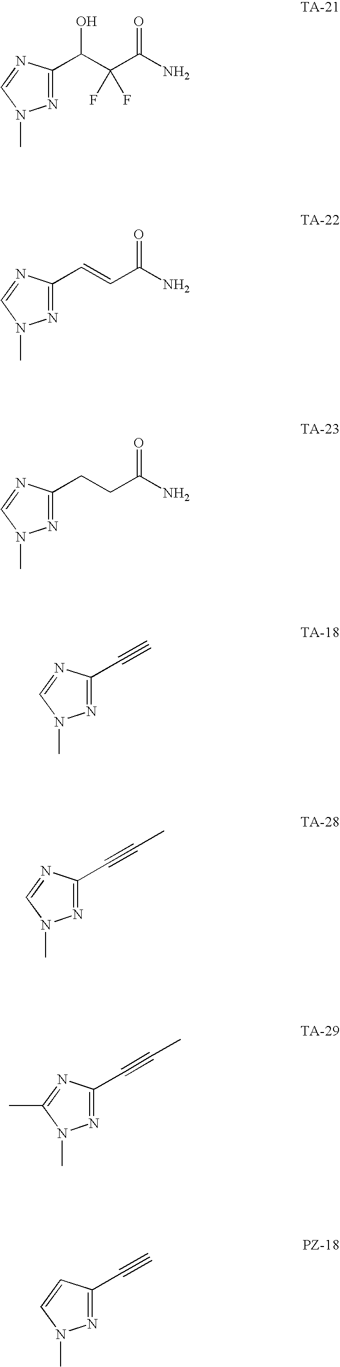

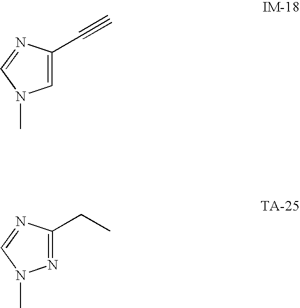

### Compound Synthesis

Compounds of the present disclosure can be prepared according to the following schemes.

### [IA-3] N¹-(3-fluorophenyl)-inosine

Reaction Scheme for the Synthesis of TBS-IA-3 and IA-3

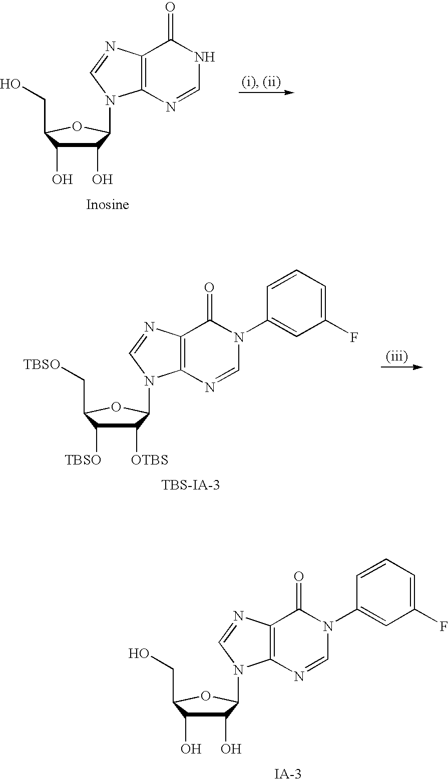

(i) TBS-Cl, imidazole, DMAP, DMF r.t 24 hrs.
(ii) 3-fluorophenylboronic acid, Cu₂(OAc)₂, pyridine, pyridine-N-oxide, CH₂Cl₂, ground 4 Å mol. sieves, O₂
(iii) TBAF, THF, −10° C.

### [RN-3] 5-amino-4-N-3-fluorophenylcarboxamide-1-β-D-ribofuranosyl-1H-imidazole

Reaction Scheme for the Synthesis of RN-3

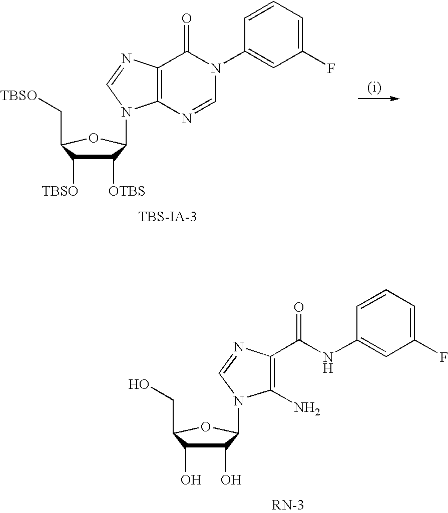

(i) 5 N NaOH, EtOH, reflux 4 hr.

The TBS-IA-3 can be prepared as disclosed above.

### [TBS-TA-8] (1-[2′,3′,5′-tris(O-tert.-butyldimethylsilyl)-β-D-ribofuranosyl]-(1,2,4-triazol-3-yl)-carboxaldehyde

Reaction Scheme for the Synthesis of TBS-TA-8^{a}

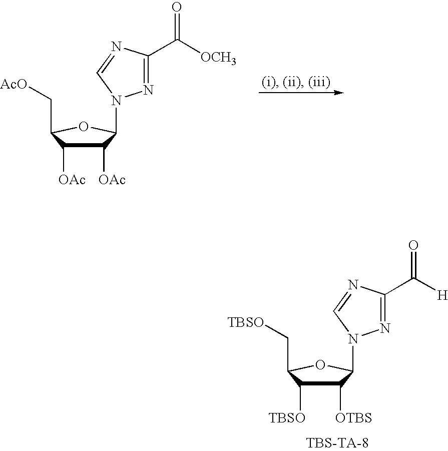

^{a}Reagents and conditions: (i) 1 M NaOMe, MeOH, room temp, 2 h; (ii) TBDMSCl, imidazole, DMAP, DMF, room temp, 18 h; (iii) DIBALH, CH₂Cl₂, −78° C., 4 h

### TA-18, 3-ethynyl-1-(β-D-ribofuranosyl)-[1,2,4]triazole

Reaction Scheme for the Synthesis of TA-18^{a}

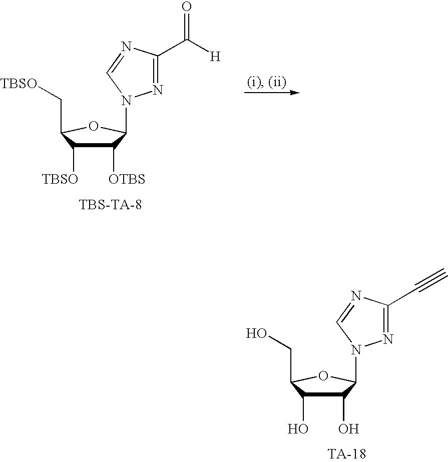

^{a}(i) dimethyl-1-diazo-2-oxopropylphosphonate, K₂CO₃, MeOH, room temp, 24 h; (ii) 1 M TBAF in THF, room temp, 2 h.

### TA-12, 1-(1-β-D-ribofuranosyl-[1,2,4]triazol-3-yl)-ethanol

Reaction Scheme for the Synthesis of TA-12^{a}

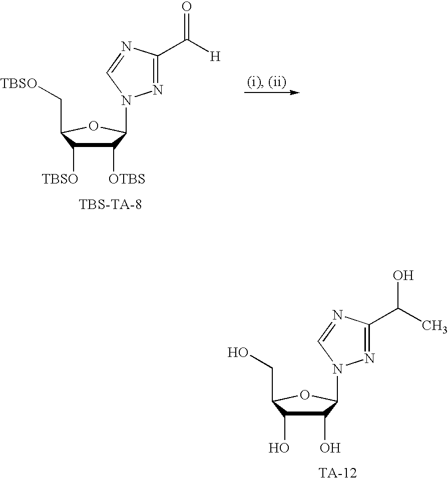

^{a}Reagents and conditions: (i) CH3MgCl, THF, 0° C., 3 h; (ii) 1 M TBAF in THF, room temp, 2 h.

### TA-13, 1-(1-β-D-ribofuranosyl-[1,2,4]triazole-3-yl)-ethanone

Reaction Scheme for the Synthesis of TA-13^{a}

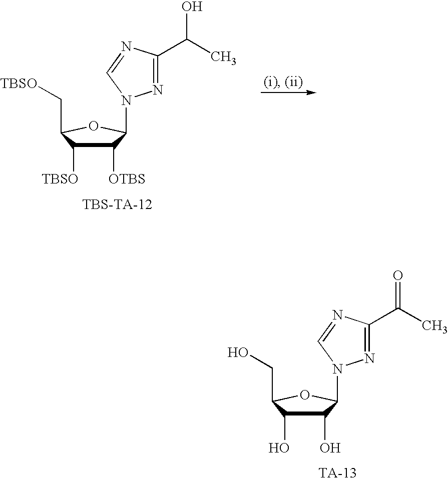

^{a}Reagents and conditions: (i) PCC, CH₂Cl₂, room temp, 4 h; (ii) 1 M TBAF in THF, room temp, 2 h.

### TA-14, 1-(1-β-D-ribofuranosyl-[1,2,4]triazol-3-yl)-phenylmethanol

Reaction Scheme for the Synthesis of TA-14^{a}

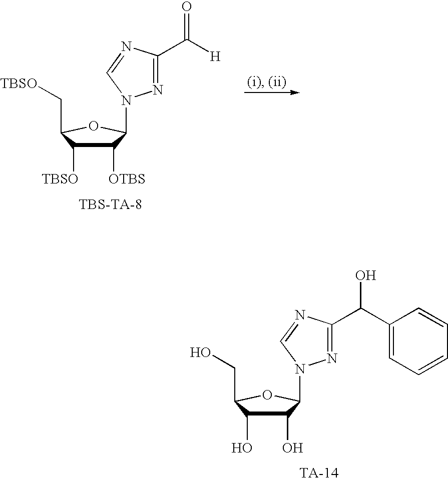

^{a}Reagents and conditions: (i) PhMgCl, THF, 0° C., 3 h; (ii) 1 M TBAF in THF, room temp, 2 h.

### TA-15, 1-(1-β-D-ribofuranosyl-[1,2,4]triazol-3-yl)-phenylmethanone

Reaction Scheme for the Synthesis of TA-15^{a}

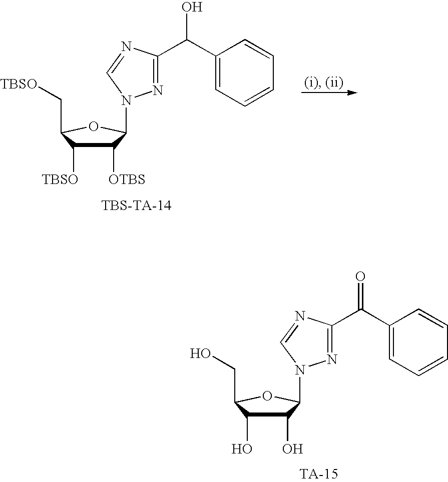

^{a}Reagents and conditions: (i) PCC, CH₂Cl₂, room temp, 4 h; (ii) 1 M TBAF in THF, room temp, 2 h.

### TA-17, 3-(1,1-difluoro-ethyl)-1-β-D-ribofuranosyl-[1,2,4]triazole

Reaction Scheme for the Synthesis of TA-17^{a}

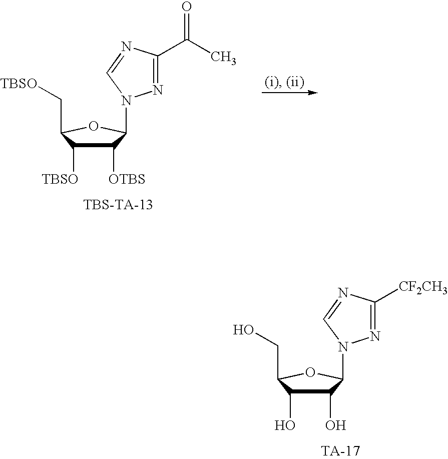

^{a}Reagents and conditions: (i) DAST, CH₂Cl₂, reflux, 12 h; (ii) 1 M TBAF in THF, room temp, 2 h.

### TA-19, 1-(1-β-D-ribofuranosyl-[1,2,4]triazol-3-yl)-2,2,2-trifluoroethanol

Reaction Scheme for the Synthesis of TA-19^{a}

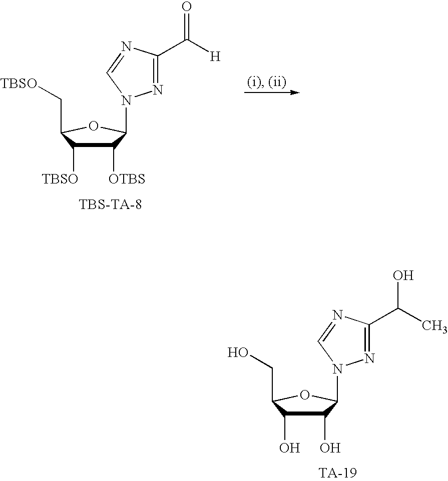

^{a}Reagents and conditions: (i) CF₃TMS, KOtBu, dry TIE, 0° C., 3 h; (ii) 1 M TBAF in TIT, dry THF, r.t., 2.5 h

### TA-20, 3-(1-β-D-ribofuranosyl-[1,2,4]triazol-3-yl)-3-hydroxypropionamide

Reaction Scheme for the Synthesis of TA-20^{a}

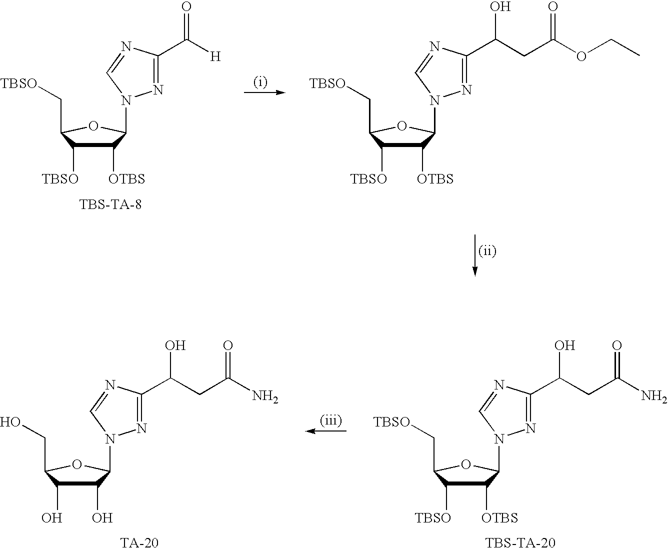

^{a}Reagents and conditions: (i) ethyl bromoacetate, Zn (m), THF, reflux, 4 h; (ii) NH₃, MeOH, 60° C., 24 h; (iii) 1 M TBAF in THF, r.t., 4 h.

The following presents various compounds along with biological test data.

### Summary of Compounds and Antiviral Activity

1-β-D-ribofuranosyl-azole derivatives compounds and screened for antiviral activity of the against Influenza A H3N2 are shown below:

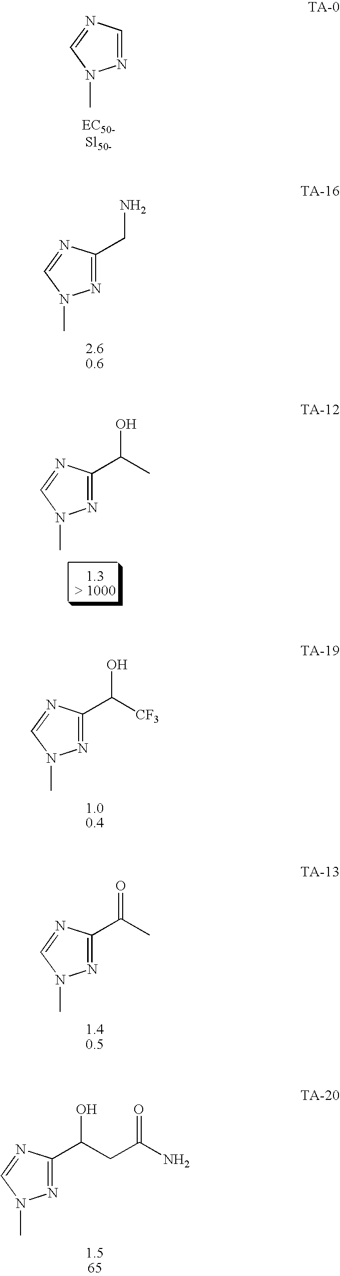

The following is a general description of the evaluation protocol used with the example being influenza virus. It being understood that the same protocol is applicable for the other viruses tested.

2.0 General Description of the Influenza Antiviral Evaluation Protocol

Antiviral and Toxicity Assay:

The influenza antiviral evaluation assay examines the effects of compounds at designated single-dose concentrations. Madin Darby canine kidney (MDCK) cells are used in the assay to test the efficacy of the compounds in preventing the cytopathic effect (CPE) induced by influenza A/Udorn/72 infection. A typical plate layout is shown below in Table 1.

TABLE 1
384-well (10 uM) plate format

| | 1 | 2 | 3 | 4 | 5 | 6 | 7 | 8 | 9 | 10 | 11 | 12 |
|---|---|---|---|---|---|---|---|---|---|---|---|---|
| A | C | 1 | 2 | 3 | 4 | 5 | 6 | 7 | 8 | 9 | 10 | 11 |
| B | C | 23 | 24 | 25 | 26 | 27 | 28 | 29 | 30 | 31 | 32 | 33 |
| C | C | 45 | 46 | 47 | 48 | 49 | 50 | 51 | 52 | 53 | 54 | 55 |
| D | C | 67 | 68 | 69 | 70 | 71 | 72 | 73 | 74 | 75 | 76 | 77 |
| E | C | 89 | 90 | 91 | 92 | 93 | 94 | 95 | 96 | 97 | 98 | 99 |
| F | C | 111 | 112 | 113 | 114 | 115 | 116 | 117 | 118 | 119 | 120 | 121 |
| G | C | 133 | 134 | 135 | 136 | 137 | 138 | 139 | 140 | 141 | 142 | 143 |
| H | C | 155 | 156 | 157 | 158 | 159 | 160 | 161 | 162 | 163 | 164 | 165 |
| I | C | 177 | 178 | 179 | 180 | 181 | 182 | 183 | 184 | 185 | 186 | 187 |
| J | C | 199 | 200 | 201 | 202 | 203 | 204 | 205 | 206 | 207 | 208 | 209 |
| K | C | 221 | 222 | 223 | 224 | 225 | 226 | 227 | 228 | 229 | 230 | 231 |
| L | C | 243 | 244 | 245 | 246 | 247 | 248 | 249 | 250 | 251 | 252 | 253 |
| M | C | 265 | 266 | 267 | 268 | 269 | 270 | 271 | 272 | 273 | 274 | 275 |
| N | C | 287 | 288 | 289 | 290 | 291 | 292 | 293 | 294 | 295 | 296 | 297 |
| O | C | 309 | 310 | 311 | 312 | 313 | 314 | 315 | 316 | 317 | 318 | 319 |
| P | C | 331 | 332 | 333 | 334 | 335 | 336 | 337 | 338 | 339 | 340 | 341 |

| | 13 | 14 | 15 | 16 | 17 | 18 | 19 | 20 | 21 | 22 | 23 | 24 |
|---|---|---|---|---|---|---|---|---|---|---|---|---|
| A | 12 | 13 | 14 | 15 | 16 | 17 | 18 | 19 | 20 | 21 | 22 | CC |
| B | 34 | 35 | 36 | 37 | 38 | 39 | 40 | 41 | 42 | 43 | 44 | CD |
| C | 56 | 57 | 58 | 59 | 60 | 61 | 62 | 63 | 64 | 65 | 66 | CD |
| D | 78 | 79 | 80 | 81 | 82 | 83 | 84 | 85 | 86 | 87 | 88 | CD |
| E | 100 | 101 | 102 | 103 | 104 | 105 | 106 | 107 | 108 | 109 | 110 | CD |
| F | 122 | 123 | 124 | 125 | 126 | 127 | 128 | 129 | 130 | 131 | 132 | CD |
| G | 144 | 145 | 146 | 147 | 148 | 149 | 150 | 151 | 152 | 153 | 154 | CD |
| H | 166 | 167 | 168 | 169 | 170 | 171 | 172 | 173 | 174 | 175 | 176 | CD |
| I | 188 | 189 | 190 | 191 | 192 | 193 | 194 | 195 | 196 | 197 | 198 | VC |
| J | 210 | 211 | 212 | 213 | 214 | 215 | 216 | 217 | 218 | 219 | 220 | VC |
| K | 232 | 233 | 234 | 235 | 236 | 237 | 238 | 239 | 240 | 241 | 242 | VC |
| L | 254 | 255 | 256 | 257 | 258 | 259 | 260 | 261 | 262 | 263 | 264 | VC |
| M | 276 | 277 | 278 | 279 | 280 | 281 | 282 | 283 | 284 | 285 | 286 | VC |
| N | 298 | 299 | 300 | 301 | 302 | 303 | 304 | 305 | 306 | 307 | 308 | VC |
| O | 320 | 321 | 322 | 323 | 324 | 325 | 326 | 327 | 328 | 329 | 330 | VC |
| P | 342 | 343 | 344 | 345 | 346 | 347 | 348 | 349 | 350 | 351 | 352 | VC |

CC = cell control.
CD positive control compound wells.
VC = virus control.
Numbers indicate individual compounds in each well.

Ribavirin is included in each run as a positive control compound. Subconfluent cultures of MDCK cells are plated into 384-well plates for the analysis of antiviral activity (CPE). Drugs are added to the cells 24 hours later. At a designated time, the CPE wells also receive 100 tissue culture infectious doses (100 TCID50s) of A/Udorn/72, 72 hours liter the cell viability is determined using CellTiter-Glo (Promega). Effective compounds are those that inhibit viral-induced CPE by more that 50%.

CellTiter-Glo Detection Assay for Cell Viability

Measurement of influenza-induced CPE is based on quantitation of ATP, an indicator of metabolically active cells. The CPE assay employs a commercially available CellTiter-Glo® Luminescent Cell Viability Kit (ProMega, Madison, Wisc.), and is a reliable method for determining cytotoxicity and cell proliferation in culture. The procedure involves adding the single reagent (CellTiter-Glo® Reagent) directly to previously cultured, subconfluent cells in media. This induces cell lysis and the production of a bioluminescent signal (half-life greater than 5 hours, depending on the cell type) that is proportional to the amount of ATP present (which is a biomarker for viability).

3.0 Materials and Methods

3.1 Materials
Cells
   MDCK, ATCC Cat # CCL-34
Virus
   A/Udorn/72; H3N2; Passage #2; 14 Oct. 05
Endpoint Reagent
   CellTiter-GLO—Promega
      Substrate—Cat #G755B
      Buffer—Cat #G756B
Control drug
   Ribavirin—MP Biomedicals, Inc., Cat #196066

3.2 Methods

On day one, MDCK cells are grown to 90% confluency, then trypsinized, recovered, centrifuged, and washed twice in PBS to remove residual serum. Afterward, the cells are diluted in serum-free DMEM, aliquoted into 384-well plates (20 ul/well), and allowed to attach to the plate overnight at 37° C.

On day two, a visual observation of cell morphology is made on a small, random sampling of plates. The tested compounds (5 ul) are added to the individual plate wells to a final concentration of 10 uM and a DMSO concentration of <0.5%. The plates are

Further details concerning the evaluation protocol can be found in Noah et al. A cell-based luminescence assay is effective for high-throughput screening of potential influenza antivirals, Antiviral Research (2006), doi:10.1016/j.antiviral.2006.07.006, (copy available on line www.sciencedirect.com), entire disclosure of which is incorporated herein by reference.

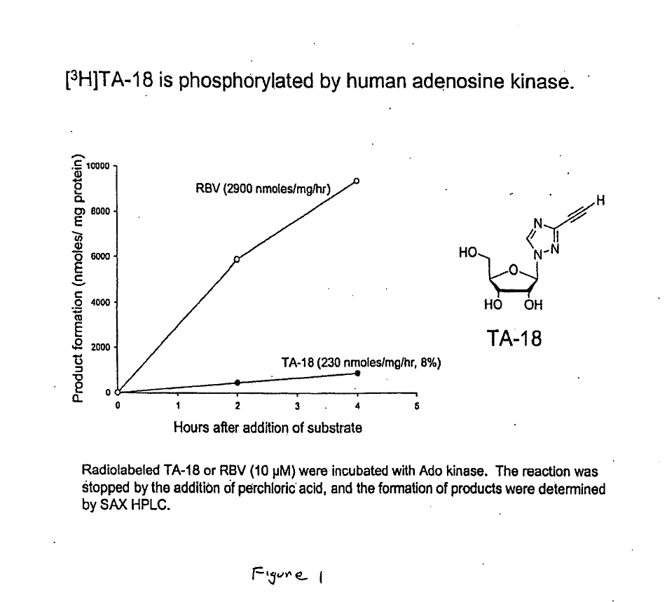

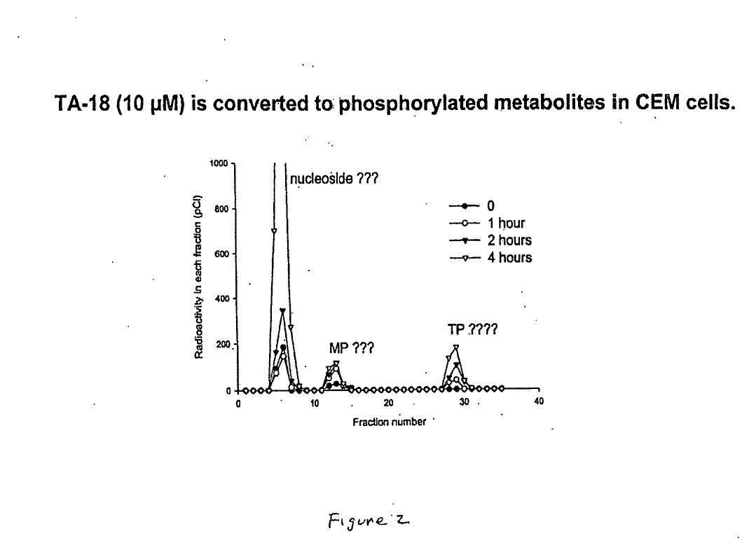

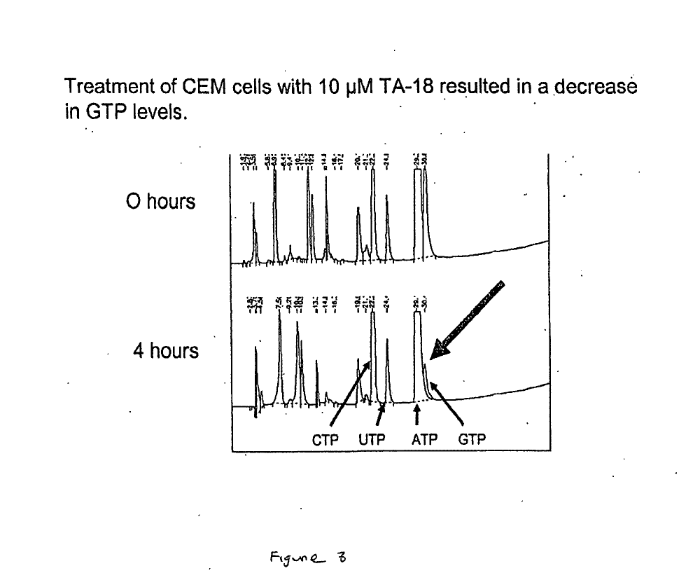

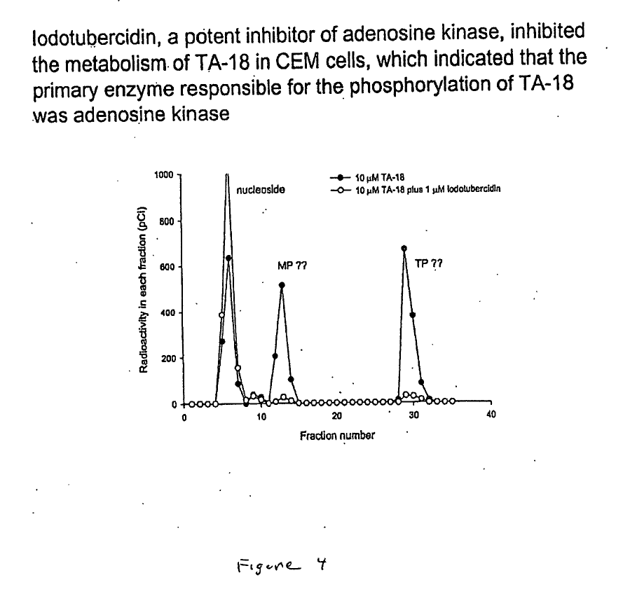

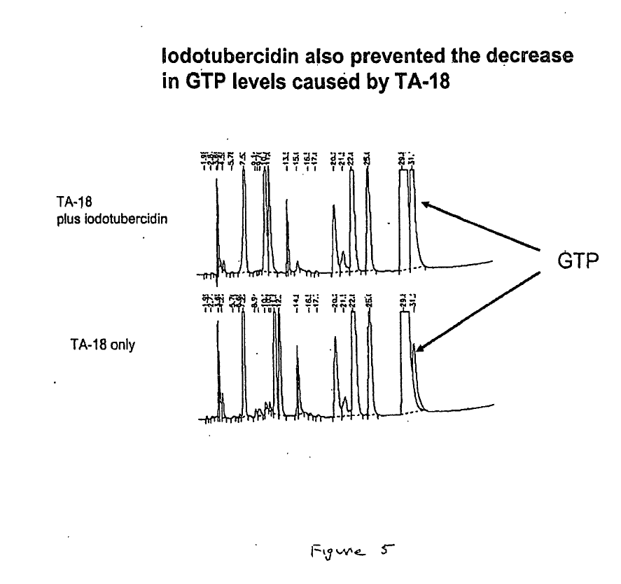

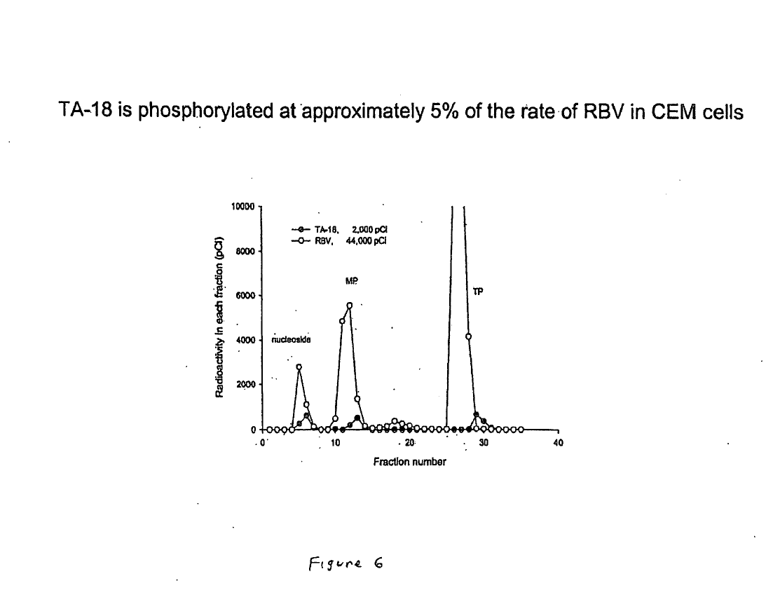

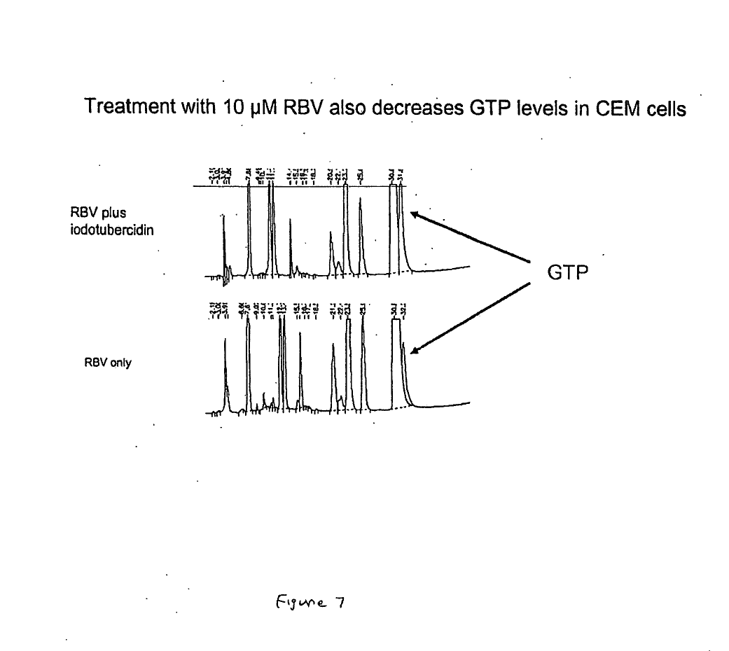

The following conclusions can be drawn from the preliminary studies with adenosine kinase and TA-18.
1. Substrate activity with adenosine kinase was determined with a number of the analogs that were synthesized (See table 2 below).
2. Using radiolabeled TA-18, it was confirmed that it is a substrate for human adenosine kinase (See FIG. 1). The discrepancy in the activity between the results shown in the table on the next page and the results with radiolabeled compound is likely due to use of different concentrations of compounds in the experiments (100 μwas used in the results shown in the Table and 10 μM was used in all the other experiments).
3. TA-18 was converted to phosphorylated metabolites in human cells (See FIG. 2).
4. Treatment with TA-18 resulted in a decline in GTP levels (See table 3 below and FIG. 3) in human cells.
5. Inhibition of adenosine kinase activity with iodotubercidin (See FIG. 4) inhibited the metabolism of TA-18 in human cells, which indicated that adenosine kinase was the primary enzyme involved in the metabolism of TA-18 in this cell line.
6. Inhibition of adenosine kinase activity with iodotubercidin (See FIG. 5) also prevented the decline in GTP levels caused by TA-18, which indicated that a metabolite of TA-18 was responsible for the decrease in GTP levels that was observed in cells treated with TA-18.
7. Treatment with ribavirin also caused a decrease in GTP levels in human cells (See FIG. 7). Since there were much less intracellular metabolites from TA-18 than from ribavirin (See FIG. 6), this result indicates that the TA-18 metabolites are more potent in reducing GTP levels than the ribavirin metabolites.
8. These preliminary results suggest that the antiviral mechanism of action of TA-18 is due to a decline in intracellular GTP levels, possibly due to the inhibition of IMP dehydrogenase activity.

TABLE 2
Adenosine kinase activity with selected nucleoside analogs

| Compound | Activity as a percent of ribavirin |
|---|---|
| RA-1 | 3.4 |
| RA-9 | <0.002 |
| TA-3 | 2.5 |
| TA-7 | <2.5 |
| TA-10 | 52 |
| TA-13 | 8 |
| TA-18 | 25 |
| TA-20 | 5 |

Human adenosine kinase was incubated with 100 μM of each compound and ATP. After incubation for the desired time at 37° C. the reaction was stopped and the conversion compound to the respective 5′-monophosphate was determined using HPLC.

In testing of these compounds, we did not distinct differences in the level of inhibition across these three and influenza. The following points highlight finding made in testing compounds of this disclosure for antiviral activity. For example, the antiviral screening against Hantaan virus (HTNV), Crimean Congo Hemorraghic fever virus (CCHFV), Rift Valley Fever virus (RVFV) and Influenza shows selectivity of compounds of this disclosure within the Bunyaviridae family. For example, 18-0 showed antiviral activity against HTNV and influenza. IA-3 showed antiviral activity against HTNV and IM-18 showed antiviral activity against influenza. PZA-O showed antiviral activity against influenza. RC-3 showed antiviral activity against HTNV and influenza, and RN-3 showed activity against HTNV. TA-1 showed antiviral activity against CCHFV, TA2 showed antiviral activity against HTNV, TA-14 and 16 showed antiviral activity against HTNV, TA18 showed antiviral activity against HTNV, influenza and CCHFV, and TA-23 showed antiviral activity against RVFV. The T-series compounds are preferred.

The following non-limiting examples are presented to further illustrate the present disclosure.

### Example 1

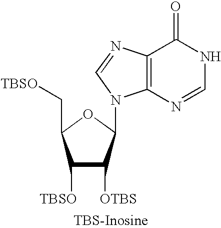

2′,3′,5′-tris-(O-tert-butyldimethylsilyl)-inosine (TBS-I): Inosine (5.36 g, 20 mmol) was protected with TBS-Cl (18.1 g, 120 mmol) and imidazole (10.9 g, 160 mmol) in dry DMF (100 mL) at r.t. for 48 h. After concentration in vacuo, the mixture was diluted with CH₂Cl₂ to 200 and washed with 100 mL portions each of water (4 washes), sat. NH₄Cl (3 washes) and sat. NaCl followed by recrystallization in EtOAc to yield a white crystalline solid (10.9 g, 17.8 mmol, 90%). FTIR (PTFE card, cm⁻¹) 1706; ¹H NMR (400 MHz, CDCl₃-d) δ13.30 (1H, s), 8.31 (1H, s), 8.21 (1H, s), 5.98 (1H, d, J=4.8 Hz), 4.46 (1H, m), 4.26 (1H, m), 4.09 (1H, m), 3.96 (1H, m), 3.75 (1H, m), 0.92-0.77 (27H, mult. s), 0.11-0.20 (18H, mult. s); ¹³C NMR (400 MHz, CDCl₃-d) δ 159.3, 148.8, 145.3, 138.8, 124.8, 88.2, 85.2, 76.4, 71.5, 62.2, TBS-not listed.

### Example 2

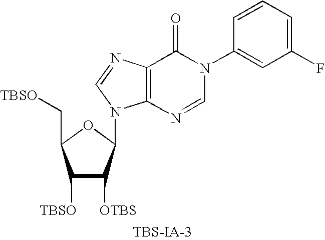

N¹-(3-fluorophenyl)-2′,3′,5′-tris-(O-tert-butyldimethylsilyl)-inosine (TBS-IA-3): To an oven dried Schlenk tube was added TBS-I (2.4 g, 4.0 mmol), 3-fluorophenylboronic acid (1.1 g, 8.0 mmol), anhydrous Cu(OAc)₂ (800.0 mg, 4.4 mmol), pyridine-N-oxide (800 mg, 4.0 mmol), ground 4 Å molecular sieves (˜1 g), and a stir bar. The tube was then sealed with a rubber septa and evacuated and flushed with oxygen. Dry pyridine (647 μL, 8.0 mmol) and molecular sieve dried CH₂Cl₂ (20 mL) were then added and the reaction was stirred vigorously at r.t. for 24 h. The reaction was then quenched with sat. NH₄OH in MeOH (0.5 mL in 5 mL respectively) followed by dilution with hexanes to 500 mL. The organics were washed with 250 mL portions of each: water, sat. NH₄Cl, 1 M NaCl, and sat. NaCl. The organics were then dried over Na₂SO₄ and concentrated in vacuo. All compounds were purified by medium pressure flash chromatography (Isco CombiFlash GRADUATE) with CH₂Cl₂ /MeOH as eluent yielding an amorphous white solid (F.W.=705.1, 1.93 g, 2.74 mmol, 67%) FTIR (PTFE card, cm⁻¹) 1716; ¹H NMR (400 MHz, CDCl₃-d) δ8.20 (1H, s), 7.99 (1H, s), 7.45 (1H, m), 7.16-7.13 (31-1, m), 5.99 (1H, d, J=4.8 Hz), 4.46 (1H, m), 4.29 (1H, m), 4.11 (1H, m), 3.97 (1H, m), 3.77 (1H, m), 0.93-0.80 (27H, mult. s), 0.12-0.16 (18H, mult. s); ¹³C NMR (400 MHz, CDCl₃-d) δ162.0 (J=248.1 Hz), 156.0, 147.1, 146.4, 138.4 (J=9.5 Hz), 130.7 (J=9.0 Hz), 124.7, 123.0, 116.3 (J=20.0 Hz), 115.2 (J=23.9 Hz), 88.1, 85.4, 76.7, 71.6, 62.3, TBS-not listed; Elem. Anal. Calcd. For C₃₄H₅₇FN₄O₅Si₃: C, 57.92; H, 8.15; N, 7.95 Found: C, 57.94; H, 8.36; N, 7.83.

### Example 3

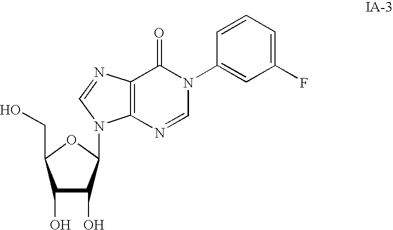

N¹-(3-fluorophenyl)-inosine (IA-3): To a round bottom flask was added TBS₃-IA-3 (1.06 g, 1.5 mmol), dry THF (25 mL), and a stir bar then set to stir at −10° C. To this was added 5.0 mL of 1M tetrabutylammonium fluoride/THF solution and after 1.5 hours (completion indicated by TLC) the solution was directly loaded a 5 cm diameter silica gel gravity column (˜350 mL of 70-230 mesh 60 Å silica gel) with acetone as eluent to remove the bulk of the tetrabutylammonium salts. The solids were then purified by medium pressure flash chromatography (Isco CombiFlash GRADUATE) with toluene/EtOH as eluent yielding an amorphous white solid (F.W.=362.3, 469 mg, 1.29 mmol, 86%) FTIR (KBr, cm⁻¹) 3394, 2931, 1699, 1601, 1578, 1546, 1489, 1226; NMR (CD₃OD, 400 MHz) δ 8.39 (1H, s), 8.30 (1H, s), 7.57 (1H, m), 7.35-7.26 (3H, m), 6.04 (1H, d, J=5.9 Hz), 4.63 (1H, m), 4.33 (1H, m), 4.13 (1H, m), 3.86 (1H, m), 3.75 (1H, m); ¹³C NMR (CD₃OD, 400 MHz) δ164.1 (J=245.4 Hz), 157.9, 149.2, 148.7, 141.5, 139.9 (J=10.2 Hz), 132.1 (J=8.7 Hz), 125.3, 124.8 (J=2.3 Hz), 117.4 (J=21.2 Hz), 116.4 (J=23.9 Hz), 90.4, 87.5, 76.3, 72.0, 62.9; MS (ESI) calcd for C₁₆H₁₅FN₄O₅ [M+1]⁺ 363.11 m/z, found 363.26 m/z.

### Example 4

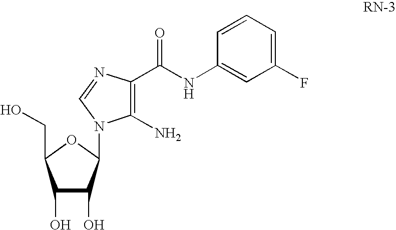

5-amino-4-N-3-fluorophenylcarboxamide-1-β-D-ribofuranosyl-1H-imidazole (RN-3): TBS-IA-3 (1.41 g, 2 mmol) was added to a round bottom flask and dissolved in absolute EtOH (30 mL) and brought to a boil while stirring. 5 N NaOH (10 mL) was then added to the solution, which was refluxed for 4 hrs. The flask was removed from the heat and cooled to r.t. then neutralized (pH=˜7) with 6 N HCl. The aqueous mixture was then extracted with 3 portions EtOAc which were subsequently dried over Na₂SO₄ and conc. in vacuo. The solids were then recrystallized in EtOAc to afford a slightly pink crystalline solid (F.W.=352.3, 450 mg, 1.28 mmol, 64%) FTIR (KBr, cm⁻¹) 3558, 3536, 3489, 3426, 3363, 3302, 3117, 2938, 2927, 1651, 1607, 1564; ¹H NMR (DMSO-d₆, 400 MHz) δ 9.57 (1H, br s), 7.79 (1H, m), 7.59 (1H, m), 7.43 (1H, s), 7.27 (1H, m), 6.77 (1H, m), 6.23 (2H, br s), 5.52 (1H, d, J=6.4 Hz), 5.44 (1H, d, J=6.4 Hz), 4.94 (1H, t, J=4.9 Hz), 4.58 (1H, d, J=5.2 Hz), 4.30 (1H, m), 4.05 (1H, m), 3.91 (1H, m) 3.59 (2H, m); ¹³C NMR (CD₃OD, 400 MHz) δ164.8, 164.3 (J=240.5 Hz), 145.9, 141.9 (J=11.0 Hz), 131.2, 131.1 (J=10.0 Hz), 115.92, 113.6, 110.5 =21.7 Hz), 107.5 (J=26.5 Hz), 90.7, 87.4, 74.0, 72.1, 62.5;MS (ESI) calcd for C₁₅H₁₇FN₄O₅ [M+1]⁺ 353.13 m/z, found 353.25 m/z.

### Example 5

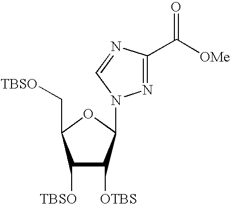

(1-[1′,3′,5′-tris(O-tert.-butyldimethylsilyl)-β-D-ribofuranosyl]-(1,2,4-triazol-3-yl)-carboxylic acid methyl ester: To a solution of methyl-1-(β-D-ribofuranosyl)-1,2,4-triazole-3-carboxylate (5.1345 g, 19.8 mmol), imidazole (10.78 g, 158.3 mmol) and DMAP (50mg) in dry DMF (50 mL), was added tert-butyldimethylsilyl chloride (11.74 g, 77.9 mmol). The reaction mixture was stirred at room temperature overnight, after which TLC analysis (5% MeOH/CH₂Cl₂, Rf=0.62) showed total conversion of starting material in a single product. The white slurry was poured in a bilayer system of water (100 mL) and DCM (100 mL). The organic layer was separated, and the aqueous phase was repeatedly extracted with DCM (3×50 mL). The combined organic extracts were dried (anhydrous Na₂SO₄), filtered, and evaporated under reduced pressure to afford a white solid, which was recrystallized from hexanes giving the desired product as a white powder (F.W. 602.00, 10.07 g, 84%). 1H NMR (200 MHz, CDCl₃) δ 8.57 (s, 1H), 5.84 (d, 1H, J_{1′,2′}=4.9 Hz, H-1′), 4.45 (m, 1H, H-2′), 4.22 (m, 1H, H-3′), 4.17-4.09 (m, 1H, H-4′), 3.99 (s, 3H), 3.98-3.90 (dd, 1H, J_{5′a,5′b}=11.9 and J_{5′a,4′}=3.7 Hz, H-5a), 3.80-3.73 (dd, 1H, J_{5′b,5′a}=11.4 and J_{5′b,4′}=2.5 Hz, H-5b), 0.94 (s, 9H, tBu), 0.91 (s, 9H, tBu), 0.85 (s, 9H, tBu), 0.13 (s, 6H, 233 CH3), 0.08 (s, 6H, 2×CH3), 0.03 (s, 3H, CH3), and −0.06 (s, 3H, CH3).

### Example 7

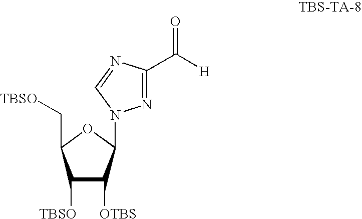

(1-[2′,3′,5′-tris(O-tert.-butyldimethylsilyl)-β-D-ribofuranosyl]-(1,2,4-triazol-3-yl)-carboxaldehyde [TBS-TA-8]: To a solution of (1-[2′,3′,5′-tris(O-tert.-butyldimethylsilyl)-β-D-ribofuranosyl]-(1,2,4-triazol-3-yl)-carboxylic acid methyl ester (4.2140 g, 7.0 mmol) in dry CH₂Cl₂ (15 mL), at −78° C. was slowly added DIBAL-H (17.5 mL, 1 M solution in CH₂Cl₂) so as to maintain the internal temperature below −65° C. The reaction was stirred for 4 h at −78° C. and then quenched by slowly adding cold (−78° C.) MeOH (7 mL) while the internal temperature was kept below −65° C. The resulting white emulsion was then allowed to come to room temp with swirling over 2 h. Then the reaction mixture was diluted by adding CH₂Cl₂ (25 mL) and washed with 0.5 M NaOH (25 mL). Then aqueous mixture was then extracted with CH₂Cl₂ (3×). The combined organic solution was washed with brine, dried over anhydrous Na₂SO₄, and concentrated under reduced pressure to give the crude product as a pale yellow oil which was then purified on a silica gel column (5% MeOH/CH₂Cl₂) to give the pure product as a colorless oil which in turn obtained as a white solid after drying under reduced pressure for 5 days (F.W. 571.97, 3.1668 g, 78%): 1H NMR (200 MHz, CDCl₃) δ 10.01(s, 1H), 8.57 (s, 1H), 5.82 (d, 1H, J_{1′,2′}=4.2 Hz, H-1′), 4.48 (m, 1H, H-2′), 4.25 (m, 1H, H-3′), 4.18-4.09 (m, 1H, H-4′), 3.95-3.88 (dd, 1H, J_{5′a,5′b}=11.9 and J_{5′a,4′}=3.7 Hz, H-5a), 3.79-3.72 (dd, 1H, J_{5′b,5′a}=11.5 and J_{5′b,4′}=2.6 Hz, H-5b), 0.92 (s, 9H, tBu), 0.91 (s, 9H, tBu), 0.84 (s, 9H, tBu), 0.10-−0.09 (mult. S, 18H).

### Example 8

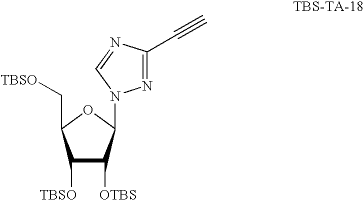

3-ethynyl-1-(2′,3′,5′-tris(O-tert-butyldimethylsilyl)-β-D-ribofuranosyl)-1,2,4-triazole [TBS-TA-18]: To a stirred solution of the (1-[2′,3′,5′-tris(O-tert.-butyldimethylsilyl)-β-D-ribofuranosyl]-(1,2,4-triazol-3-yl)-carboxaldehyde [TBS-TA-8] (572 mg, 1 mmol) and dimethyl-1 -diazo-2-oxopropylphosphonate (249 nag, 1.3 mmol) in anhydrous methanol (5 ml), was added anhydrous K₂CO₃ (208 mg, 2.1 mmol). The resulting pale yellow solution was stirred for 24 h. The mixture was quenched with water (10 ml) and extracted with Et₂O (4×20 ml). The combined extracts were washed with NaHCO_{3(aq)} (sat., 10 ml) and brine (sat, 10 ml), then dried over Na₂SO₄. Removal of solvent in vacuo afforded the crude product which was purified by flash chromatography (5%-20% EtOAC/Hexane) to yield a white solid that was recrystallized from hexanes giving the desired product as a white powder (F. W. 567.98, 435 mg, 76%); 1H NMR (200 MHz, CDCl₃) δ 8.72 (s, 1H), 5.69 (d, 1H, J_{1′,2′}=4.03 Hz, H-1′), 4.45 (m, 1H, H-2′), 4.23 (m, 1H, H-3′), 4.11 (m, 1H, H-4′), 3.95-3.88 (dd, 1H, J=5′a,5′b=11.5 and J_{5′a,4′}=4.03 Hz, H-5a), 3.79-3.72 (dd, 1H, J_{5′b,5′a}=11.35 and J_{5′b,4′}=2.9 Hz, H-5b), 3.06 (s, 1H), 0.95-0.78 (mult. s, 27H), 0.14-−0.09 (mult. s, 18H). LCMS (APCI) calcd for C₂₇H₅₃N₃O₄Si₃[M+1]⁺ 568.34 m/z, found 568.28 m/z. HPLC 100% CH₃CN, rt 6.62 min.

### Example 9

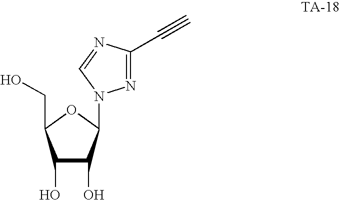

3-ethynyl-1-(β-D-ribofuranosyl)-[1,2,4]triazole [TA-18J: To a stirred solution of the 3-ethynyl-1-(2′,3′,5′-tris(O-tert.-butyldimethylsilyl)-β-D-ribofuranosyl)-1,2,4-triazole [TBS-TA-18} (125 mg, 0.22 mmol) in anhydrous THF (3 ml), was added 1 M TBAF in THF (0.8 mL, 0.8 mmol). The mixture was stirred at room temperature for 2 h, until completion of the reaction as shown by TLC (5% MeOH/CH₂Cl₂) and quenched with MeOH (2 ml). The solvent was removed under reduced pressure, and the product was isolated by flash chromatography (50%-Acetone/CH₂Cl₂) to yield a white solid which was recrystallized from (5% MeOH/CH₂Cl₂) to afford the desired product as a white crystalline powder (F. W. 225.20, 41 mg, 82%); 1H NMR (200 MHz, CD₃OD) δ 8.72 (s, 1H), 5.84 (d, 1H, J_{1′,2′}=3.5 Hz, H-1′), 4.43 (m, 1H, H-2′), 4.29 (m, 1H, H-3′), 4.09 (m, 1H, H-4′), 3.83-3.79 (dd, 1H, J_{5′a,5′b}=12.3 and J_{5′a,4′}=3.3 Hz, H-5a), 3.73 (s, 1H), 3.70-3.65 (dd, 1H, J_{5′b,5′a}=12.9 and J_{5′b,4′}=4.7 Hz, H-5b). ¹³C NMR (CD₃OD, 400 MHz) δ148.4, 145.6, 93.9, 86.9, 80.3, 75.0, 76.5, 71.6, 62.8. LCMS (ESI) calcd for C₉H₁₁N₃O₄ [M+1]⁺ 226.08 m/z, found 225.23 m/z.

### Example 10

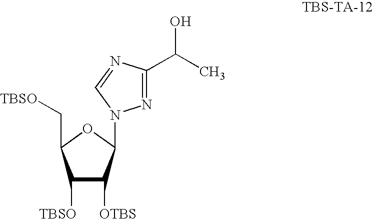

1-(2′,3′,5′-tris(O-tert.-butyldimethylsilyl)-1-β-D-ribofuranosyl-[1,2,4]triazol-3-yl)-ethanol [TBS-TA-12]: To a solution of (1-[2′,3′,5′-tris(O-tert.-butyldimethylsilyl)-β-D-ribofuranosyl]-(1,2,4-triazol-3-yl)-carboxaldehyde [TBS-TA-8] under argon (1.1420 g, 2 mmol) in THF (50 mL) at 0° C., was added CH₃MgCl (1.35 mL, 3 M solution in THF) in a dropwise manner. The reaction mixture was stirred and progress of the reaction was monitored by TLC (5% MeOH/CH₂Cl₂, Rf=0.3). Complete disappearance of the starting material was observed after 3 h. The reaction mixture was then quenched with sat. NH₄Cl_{(aq) (}20 mL) and extracted with diethyl ether (3×25 mL). The combined organic extracts were dried (anhydrous Na₂SO₄), filtered, and evaporated under reduced pressure to afford a colorless oil, which was purified on a silica gel column (5% MeOH/CH₂Cl₂) to give the product as a colorless oil. (F.W. 588.02, 1.0216 g, 87%).

### Example 11

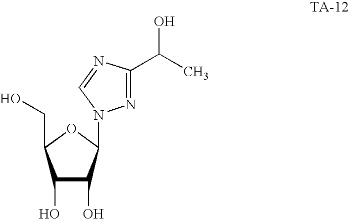

To a stirred solution of 1-(2′,3′,5′-tris(O-tert.-butyldimethylsilyl)-1-β-D-ribofuranosyl-[1,2,4]triazol-3-yl)-ethanol [TBS-TA-12] (392 mg, 0.67 mmol) in anhydrous THF (3 ml), was added 1 M TBAF in THF (0.8 mL, 0.8 mmol). The mixture was stirred at room temperature for 2 h, until completion of the reaction as shown by TLC (5% MeOH/CH₂Cl₂) and quenched with MeOH (2 ml). The solvent was removed under reduced pressure, and the product was isolated by flash chromatography (50%-Acetone/CH₂Cl₂) to yield colorless oil. (F. W. 245.10, 130 mg, 79%); 1H NMR (200 MHz, CD₃OD) δ 8.63 (s, 1H), 5.82 (d, 1H, J_{1′,2′}=3.91 Hz, H-1′), 4.89 (q, 1H, J_{′}=6.64 Hz), 4.45 (m, 1H, H-2′), 4.32 (m, 1H, H-3′), 4.08 (m, 1H, H-4′), 3.83-3.79 (dd, 1H, J_{5′a,5′b}=12.1 and J_{5′a,4′}=3.1 Hz, H-5a), 3.79-3.72 (dd, 1H, J_{5′b,5′a}=12.30 and J_{5′b,4′}=4.5 Hz, H-5b), 1.52 (d, 3H, J_{′}=6.64 Hz). ¹³C NMR (CD₃OD, 400 MHz) δ168.4, 145.6, 93.4, 86.9, 76.4, 71.8, 64.8, 63.1, 22.5. LCMS (ESI) calcd for C₉H₁₅N₃O₅ [M+1]⁴ 246.11 m/z, found 246.20 m/z.

### Example 12

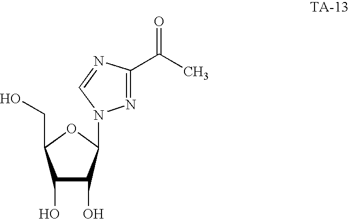

1-(1-β-D-ribofuranosyl-[1,2,4]triazole-3-yl)-ethanone [TA-13]: To a suspension of 1-(2′,3′,5′-tris(O-tert.-butyldimethylsilyl)-1-β-D-ribofuranosyl-[1,2,4]triazol-3-yl)-ethanol [TBS-TA-12] (1.764 g, 3 mmol) and ground mol. sieves (0.3 g) under argon in CH₂Cl₂ (15 mL) was added PCC (0.970 g, 4.5 mmol) and stirred under room temp while monitoring the progress of the reaction by TLC (5% MeOH/CH₂Cl₂, Rf=0.7). Complete disappearance of the starting material was observed after 4 h. The reaction mixture was then filtered through fluorosil and concentrated under reduced pressure. The resulting residue was then partitioned between water and diethyl ether, and extracted with diethyl ether (3×25 mL). The combined organic extracts were dried (anhydrous Na₂SO₄), filtered, and evaporated under reduced pressure, and the product was isolated by flash chromatography (1% MeOH/CH₂Cl₂) as a white solid. (F.W. 586.00, 1.102 g, 62%). This product (207 mg, 0.35 mmol) was then dissolved in anhydrous THF (3 ml), was added 1 M TBAF in THF (1 mL, 1 mmol). The mixture was stirred at room temperature for 2 h, until completion of the reaction as shown by TLC (5% MeOH/CH₂Cl₂) and quenched with MeOH (2 ml). The solvent was removed under reduced pressure, and the product was isolated by flash chromatography (50%-Acetone/CH₂Cl₂) to yield the desired product as a white solid (F. W. 243.22, 65 mg, 76%); 1H NMR (200 MHz, CD₃OD) δ 8.84 (s, 1H), 5.94 (d, 1H, J_{1′,2′}=3.30 Hz, H-1′), 4.49 (m, 1H, H-2′), 4.35 (m, 1H, H-3′), 4.13 (m, 1H, H-4′), 3.88-3.81 (dd, 1H, J_{5′a,5′b}=12.1 and J_{5′a,4′}=3.3 Hz, H-5a), 3.74-3.66 (dd, 1H, J_{5′b,5′a}=12.10 and J_{5′b,4′}=4.4 Hz, H-5b), 2.61 (s, 3H). LCMS (ESI) calcd for C₉H₁₃N₃O₅ [M+1]⁺ 244.09 m/z, found 244.25 m/z.

### Example 13

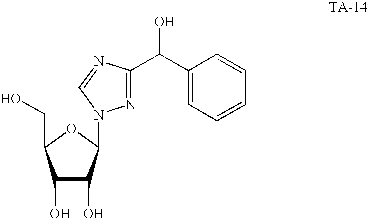

1-(1-β-D-ribofuranosyl-[1,2,4]triazol-3-yl)-phenylmethanol [TA-14]: To a solution of (1-[2′,3′,5′-tris(O-tert.-butyldimethylsilyl)-β-D-ribofuranosyl]-(1,2,4-triazol-3-yl)-carboxaldehyde [TBS-TA-8] under argon (320 mg, 0.56 mmol) in THF (2 mL) at 0° C., was added PhMgCl (0.56 mL, 2 M solution in THF) in a dropwise manner. The reaction mixture was stirred and progress of the reaction was monitored by TLC (5% MeOH/CH₂Cl₂, Rf=0.33). Complete disappearance of the starting material was observed after 2 h. The reaction mixture was then quenched with sat. NH₄Cl_{(sq)} (20 mL) and extracted with diethyl ether (3×25 mL). The combined organic extracts were dried (anhydrous Na₂SO₄), filtered, and evaporated under reduced pressure to afford a colorless crude product as an oil, which was purified by flash chromatography (5% MeOH/CH₂Cl₂) to give 1-(2′,3′,5′-tris(O-tert-butyldimethylsilyl)-1-β-D-ribofuranosyl-[1,2,4]triazol-3-yl)-phenylmethanol [TBS-TA-14] as a colorless oil. (F.W. 650.08, 269 mg, 74%).

To a stirred solution of TBS-TA-14 (195 mg, 0.3 mmol) in anhydrous THF (3 ml), was added 1 M TBAF in THF (1 mL, 1 mmol). The mixture was stirred at room temperature for 2 h, until completion of the reaction as shown by TLC (5% MeOH/CH₂Cl₂) and quenched with MeOH (2 ml). The solvent was removed under reduced pressure, and the product was isolated by flash chromatography (50%-Acetone/CH₂Cl₂) to yield the product as a colorless oil (F. W. 307.30, 68 mg, 74%); 1H NMR (400 MHz, CD₃OD, complicated mixture of diast.) δ 8.62 (s, 1H), 7.49-7.23 (m, 5H), 5.82 (d, 1H, J_{1′2′}=3.71 Hz, H-1′), 4.45 (m, 1H), 4.31 (m, 1H), 4.07 (m, 1H), 3.82-3.59 (m, 2H), 2.31 (s, 1H). LCMS (APCI) calcd for C₁₄H₁₇N₃O₅[M+1]⁺ 308.12 m/z, found 308.24 m/z.

### Example 14

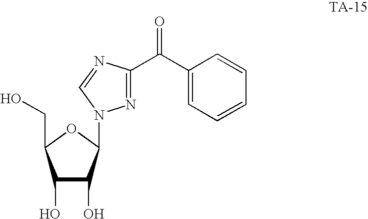

1-(1-β-D-ribofuranosyl-[1,2,4]triazol-3-yl)-phenylmethanone [TA-15]: To a suspension of 1-(2′,3′,5′-tris(O-tert.-butyldimethylsilyl)-1-β-D-ribofuranosyl-[1,2,4]triazol-3-yl)-phenylmethanol [TBS-TA-14] [TBS-14] (0.749 g, 1.15 mmol) and ground mol. sieves (0.2 g) under Ar in CH₂Cl₂ (5 mL) was added PCC (0.373 g, 1.73 mmol) and stirred under room temp while monitoring the progress of the reaction by TLC (5% MeOH/CH₂Cl₂, Rf=0.75). Complete disappearance of the starting material was observed after 4 h. The reaction mixture was then filtered through fluorosil and concentrated under reduced pressure. The resulting residue was then partitioned between water and diethyl ether, and extracted with diethyl ether (3×25 mL). The combined organic extracts were dried (anhydrous Na₂SO₄), filtered, and evaporated under reduced pressure to give the crude product as a white solid. (F.W. 305.29, 0.5021 g, 67%). This product (0,198 g, 0.3 mmol) was then dissolved in anhydrous THF (3 ml), was added 1 M TBAF in THF (1 mL, 1 mmol). The mixture was stirred at room temperature for 2 h, until completion of the reaction as shown by TLC (5% MeOH/CH₂Cl₂) and quenched with MeOH (2 ml). The solvent was removed under reduced pressure, and the product was isolated by flash chromatography (Acetone) to yield the desired product as a white solid (F. W. 305.29, 90 mg, 98%); 1H NMR (200 MHz, D₂O) δ 8.82 (s, 1H), 8.10 (m, 2H), 7.72 (m, 1H), 7.56 (m, 1H), 6.08 (d, 1H, J_{1′,2′}=3.30 Hz, H-1′), 4.62 (m, 1H, H-2′), 4.44 (m, 1H, H-3′), 4.19 (m, 1H, H-4′), 3.88-3.80 (dd, 1H, J_{5′a,5′b}=12.82 and J_{5′a,4′}=3.3 Hz, H-5a), 3.74-3.65 (dd, 1H, J_{5′b,5′a}=12.82 and J_{5′b,4′}=5.1 Hz, H-5b). LCMS (ESI) calcd for C₉H₁₃N₃O₅ [M+1]⁺ 306.11 m/z, found 306.29 m/z.

### Example 15

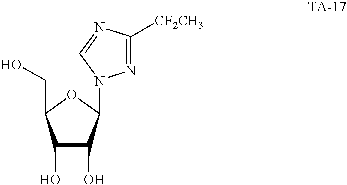

3-(1,1-difluoro-ethyl)-1-β-D-ribofuranosyl-[1,2,4]triazole [TA-17]: To a solution of 1-(2′,3′,5′-tris(O-tert-butyldimethylsilyl)-β-D-ribofuranosyl-[1,2,4]triazole-3-yl)-ethanone [TBS-TA-13] (87 mg, 0.14 mmol) in CH₂Cl₂ (5 mL) was added DAST (20 μL, 0.16 mmol) and refluxed while progress of the reaction was monitored by TLC (5% MeOH/CH₂Cl₂, Rf=0.7). After 12 h, the reaction mixture was quenched with H₂O (25 mL) in a dropwise manner, CH₂Cl₂ (25 mL) was added, the organic layer was separated and washed with saturated NaHCO₃ and H₂O (3×25 mL). The organic layer was then dried (anhydrous Na₂SO₄), filtered, and evaporated under reduced pressure, and the product TBS-TA-17 was isolated by flash chromatography (5% MeOH/CH₂Cl₂) as a white solid. (F.W. 608, 36.4 mg, 42%).

A solution of 1 M TBAF in THF (0.2 mL, 1 mmol) was added to a solution of TBS-TA-17 (36.4 mg, 0.06 mmol) in anhydrous THF (3 mL). The mixture was stirred at room temperature for 2 h, until completion of the reaction as shown by TLC (5% MeOH/CH₂Cl₂) and quenched with MeOH (2 mL). The solvent was removed under reduced pressure, and the product was isolated by flash chromatography (50%-Acetone/CH₂Cl₂) to yield the desired product as a white solid (F. W. 265.21, 12 mg, 75%); 1H NMR (400 MHz, CD₃OD) (δ 8.79 (s, 1H), 5.88 (d, 1H, J_{1′,2′}=3.52 Hz, H-1′), 4.46 (m, 1H, H-2′), 4.32 (m, 1H, H-3′), 4.10 (m, 1H, H-4′), 3.88-3.81 (dd, 1H, J_{5′a,5′b}=12.1 and J_{5′a,4′}=3.5 Hz, H-5a), 3.74-3.66 (dd, 1H, J_{5′b,5′a}=12.1 and J_{5′b,4′}=4.7 Hz, H-5b), 2.61 (t, 3H, J=18.5 Hz).

### Example 16

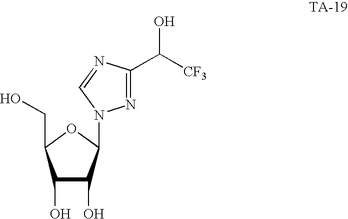

1-(1-β-D-ribofuranosyl-[1,2,4]triazol-3-yl)-2,2,2-trifluoroethanol [TA-19]: To a solution of (1-[2′,3′,5′-tris(O-tert.-butyldimethylsilyl)-β-D-ribofuranosyl)-(1,2,4-triazol-3-yl)-carboxaldehyde [TBS-TA-8] (100 mg, 0.17 mmol) in dry THF (2 mL), at 0° C. was added trimethyl(trifluoromethyl)silane (33 μL, 0.21 mmol) and catalyst KO^{t}Bu (1 mg). The reaction was stirred at this temperature under an argon atmosphere for 4.5 h. The reaction mixture was evaporated at room temperature, the oily residue was dissolved in ether (4 mL), washed with water (2 mL), dried over anhydrous Na₂SO₄ and solvent evaporated. The crude product was purified on a silica gel column (mobile phase gradient ethyl acetate in hexanes 10% to 20%) to give the pure product TBS-TA-19 as colorless oil (94 mg, 86%). FT-IR (NaCl, cm⁻¹) 2955, 2931, 1473, 1258, 1172, 1136, 837, 779. ¹H NMR (200 MHz, CDCl₃) δ 8.35 (s, 1H), 5.75 (d, 1H, J=4.9 Hz), 5.16 (q, 1H, J=6.6 Hz), 4.49-4.58 (m, 1H), 4.21-4.26 (m, 1H), 4.06-4.13 (m, 1H), 3.82-3.91 (m, 1H), 3.67-3.76 (m, 1H), 0.92 (s, 18H), 0.83 (s, 9H), 0.14 (s, 3H), 0.13 (s, 6H), 0.09 (s, 6H), 0.01 (s, 3H). ¹³C NMR (100 MHz, CDCl₃) δ 158.69, 144.34, 123.48 (q, J=282 Hz), 91.91, 86.17, 76.10, 71.90, 67.63 (q, J=34 Hz), 62.47, 25.97 (3C), 25.78 (3C), 25.61 (3C), 18.43, 18.01, 17.89, −4.51, −4.69 (2C), −5.40, −5.51 (2C).

To a stirred solution of TBS-TA-19 (434 mg, 0.68 mmol) in anhydrous THF (3 mL), was added 1 M TBAF in THF (1.4 mL, 1.4 mmol). The mixture was stirred at room temperature for 2.5 h and quenched with MeOH (1 ml). The solvent was removed under reduced pressure, and the product was isolated by flash chromatography (30% acetone 70% hexanes) to yield desired product as an oil (95 mg, 47%). FT-IR (NaCl, cm⁻¹) 3350, 1660, 1524, 1270, 1183, 1134, 867. ¹H NMR (200 MHz, CDCl₃) δ 8.66 (s, 1H), 5.86 (d, 1H, J=3.3 Hz), 5.24 (q, 1H, J=7.0 Hz) 4.50 (m, 1H), 4.37 (m, 1H), 4.07 (m, 1H), 3.77 (dd, 1H, J₁=11.9 Hz, J₁=3.3 Hz), 3.72 (dd, 1H, J₁=11.9 Hz, J₁=3.3 Hz). ¹³C NMR (100 MHz, CDCl₃) δ 160.11, 145.55, 125.10 (q, J=282 Hz), 93.29, 86.94, 76.41, 71.59, 68.08 (q, J=33 Hz), 62.75.

### Example 17

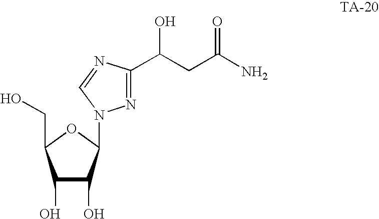

3-(1-β-D-ribofuranosyl-[1,2,4]triazol-3-yl)-3-hydroxypropionamide [TA-20]: Zinc metal was washed with EtOH, acetone and ether and dried. Zn (130 mg, 2.1 mmol) was then added to a solution of (1-[2′,3′,5′-tris(O-tert.-butyldimethylsilyl)-β-D-ribofuranosyl]-(1,2,4-triazol-3-yl)-carboxaldehyde [TBS-TA-8] (572 mg, 1.0 mmol), ethyl bromoacetate (0.35 ml, 3.1 mmol) in THF (10 ml). The reaction mixture was refluxed for 3.5 h. The solution was then diluted with CH₂Cl₂ (25 ml) then washed with 3 portions of water, dried over Na₂SO₄ and concentrated under reduced pressure. The resulting oil was purified on a silica gel column (10-20% EtOAc/Hexanes) to yield the pure product 3-(2′,3′,5′-O-tris(tert-butyldimethylsilyl)-β-D-ribofuranosyl)-[1,2,4]triazol-3-yl)-3-hydroxypropanoic acid ethyl ester (F.W. 660.08, 455 mg, 69%). This material was used in the subsequent step.

In a pressure tube MeOH (15 ml) was saturated with gaseous NH₃ and 3-(2′,3′,5′-O-tris(tert-butyldimethylsilyl)-β-D-ribofuranosyl)-[1,2,4]triazol-3-yl)-3-hydroxypropanoic acid ethyl ester (306 mg, 0.46 mmol) was added to the solution. The reaction was heated at 60° C. for 24 h. The resulting solution was concentrated under reduced pressure. The crude product was purified by flash chromatography (20-40% EtOAc/Hexanes) to yield 3-(2′,3′,5′-O-tris(tert-butyldimethylsilyl)-β-D-ribofuranosyl)-[1,2,4]triazol-3-yl)-3-hydroxypropionamide. (F.W. 631.04, 240 mg, 88%). This material was used in the subsequent step.

The 3-(2′,3′,5′-O-tris(tert-butyldimethylsilyl)-β-D-ribofuranosyl)-[1,2,4]triazol-3-yl)-3-hydroxypropionamide (150 mg, 0.24 mmol) was combined with 1M tetrabutylammonium fluoride solution (0.8 ml, 0.8mmol) in dry THF (4 ml) and stirred at r.t. for 4 h. The reaction was quenched with 5 ml of MeOH, then concentrated under reduced pressure. The crude product was purified by flash chromatography (50% EtOH/toluene); (F.W. 288.26, 24 mg, 35%); 1H NMR (200 MHz, CD₃OD) δ 8.64 (s, 1H), δ 5.83 (d, 1H, J=3.7, H-1′), δ 5.16 (m, 1H), δ 4.45, 1H, H-2′), δ 4.33 (m, 1H, H-3′), δ 4.09 (m, 1H, H-4′), δ 3.77-3.84 (dd, 1H, J_{5′a,4′}=2.9, J_{5′a,5′b}=12.1, H-5′a), δ 3.63-3.71 (dd, 1H, J_{5′b,4′}=4.8, J_{5′b,5′a}=12.8, H-5b′) δ 2.75-2.81 (m, 2H).

### Formulations

The compounds of the present disclosure can be administered by any conventional means available for use in conjunction with pharmaceuticals, either as individual therapeutic agents or in a combination of therapeutic agents. They can be administered alone, but generally administered with a pharmaceutical carrier selected on the basis of the chosen route of administration and standard pharmaceutical practice. The compounds can also be administered in conjunction with other therapeutic agents such as interferon (IFN), interferon α-2a, interferon α-2b, consensus interferon (CIFN), ribavirin, amantadine, remantadine, interleukin-12, ursodeoxycholic acid (UDCA), and glycyrrhizin.

The pharmaceutically acceptable carriers described herein, for example, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The compounds of this disclosure can be administered by any conventional method available for use in conjunction with pharmaceuticals, either as individual therapeutic agents or in a combination of therapeutic agents.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the preferred dose being 0.1 to about 30 mg/kg.

Dosage forms (compositions suitable for administration) contain from about 1 mg to about 500 mg of active ingredient per unit. In these pharmaceutical compositions, the active ingredient will ordinarily be present in an amount of about 0.5-95% weight based on the total weight of the composition.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation of a drug powder mist. Other dosage forms are potentially possible such as administration transdermally, via patch mechanism or ointment.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds of the present disclosure, alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to an animal, particularly a human, in the context of the present disclosure should be sufficient to affect a therapeutic response in the animal over a reasonable time frame. One skilled in the art will recognize that dosage will depend upon a variety of factors including a condition of the animal, the body weight of the animal, as well as the severity and stage of the condition being treated.

A suitable dose is that which will result in a concentration of the active agent in a patient which is known to affect the desired response. The preferred dosage is the amount which results in maximum inhibition of the condition being treated, without unmanageable side effects.

The size of the dose also will be determined by the route, timing and frequency of administration as well as the existence, nature, and extend of any adverse side effects that might accompany the administration of the compound and the desired physiological effect.

Useful pharmaceutical dosage forms for administration of the compounds according to the present disclosure can be illustrated as follows:

### Hard Shell Capsules

A large number of unit capsules are prepared by filling standard two-piece hard gelatine capsules each with 100 mg of powdered active ingredient, 150 mg of lactose, 50 mg of cellulose and 6 mg of magnesium stearate.

### Soft Gelatin Capsules

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

### Tablets

A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

### Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Moreover, the compounds of the present disclosure can be administered in the form of nose drops, or metered dose and a nasal or buccal inhaler. The drug is delivered from a nasal solution as a fine mist or from a powder as an aerosol.

The foregoing description of the disclosure illustrates and describes the present disclosure. Additionally, the disclosure shows and describes only the preferred embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

The embodiments described hereinabove are further intended to explain best modes known of practicing it and to enable others skilled in the art to utilize the disclosure in such, or other, embodiments and with the various modifications required by the particular applications or uses. Accordingly, the description is not intended to limit it to the form disclosed herein. Also, it is intended that the appended claims be construed to include alternative embodiments.

All publications, patents and patent applications cited in this specification are herein incorporated by reference, and for any and all purposed, as if each individual publication, patent or patent application were specifically and individually indicates to be incorporated by reference. In the case of inconsistencies, the present disclosure will prevail.

What is claimed is:

1. A compound represented by the formulae: 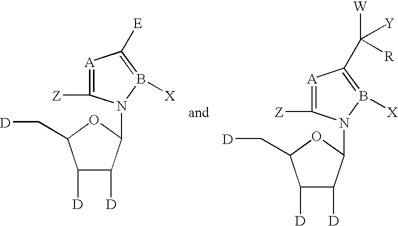
wherein A=C or N
B═C or N
X═H; C₁-C₆ alkyl, cycloalkyl, alkenyl, cycloalkenyl, alkynyl, aryl, heterocyclo, halogen such as F, Cl, Br and I; OH, NH₂, NH—(C₁-C₆ alkyl, cycloalkyl, aryl, or heterocyclo);
Z═H; C₁-C₆ alkyl, cycloalkyl, alkenyl, cycloalkenyl, alkynyl, aryl, heterocyclo, halogen, OH, NH₂, NH—(C₁-C₆ alkyl, cycloalkyl, aryl, or heterocyclo;
E=(CH₂)ₙONHR¹; n is an integer from 0-6;
R¹=aryl or heterocyclo;
each of W, Y, R is individually selected from the group consisting of H; C₁-C₆ alkyl, cycloalkyl, alkenyl, cycloalkenyl, alkynyl, aryl, heterocyclo; halogen, O, OH, Oalkyl, Oaryl, NH₂, NH—(C₁-C₆ alkyl, cycloalkyl, aryl, or heterocyclo); provided that at least one of W, Y, and R is other than H and NH₂ and wherein both W and Y together can be ═O; and
each D individually is OH, Oalkyl, Oaryl, Fl and H
; pharmaceutically acceptable salt thereof, a prodrug thereof and mixtures thereof.

2. The compound of claim 1 being N¹-(3-fluorophenyl)-inosine.

3. The compound of claim 1 being 5-amino-4-N-3-fluorophenylcarboxamide-β-D-ribofuranosyl-1H-imidazole.

4. The compound of claim 1 being 3-ethynyl-1-(β-D-ribofuranosyl)-[1,2,4]triazole.

5. The compound of claim 1 being 1-(1-β-D-ribofuranosyl-[1,2,4]triazol-3-yl)-phenylmethanol.

6. The compound of claim 1 being 1-(1-β-D-ribofuranosyl-[1,2,4]triazol-3-yl)-phenylmethanone.

7. The compound of claim 1 being 3-(1,1-difluoro-ethyl)-1-β-D-ribofuranosyl-[1,2,4]triazole.

8. The compound of claim 1 being 1-(1-β-D-ribofuranosyl-[1,2,4]triazol-3-yl)-2,2,2-trifluoroethanol.

9. The compound of claim 1 being 3-(1-β-D-ribofuranosyl-[1,2,4]triazol-3-yl)-3-hydroxypropionamide.

10. A pharmaceutical composition comprising a compound of claim 1 and a pharmaceutically acceptable carrier.

11. A method for inhibiting RNA viral polymerase in a patient by administering to the patient at least one compound according to claim 1.

12. A method for treating a patient suffering from an RNA viral infection which comprises administering to said patient an effective amount of at least one compound according to claim 1.

13. A method for treating a patient suffering from Influenza which comprises administering to said patient an effective amount of at least one compound according to claim 1.

14. A method for treating a patient suffering from Hantaan Virus which comprises administering to said patient an effective amount of at least one compound according claim 1.

15. A method for treating a patient suffering from a Crimean Congo hemorrhagic fever virus which comprises administering to said patient an effective amount of at least one compound according claim 1.

16. A method for treating a patient suffering from a Bunyaviridae family virtue which comprises administering to said patient an effective amount of at least one compound according claim 1.

17. A method for inhibiting in a patient in need thereof a RNA viral polymerase which comprises administering to said patient an effective amount of at least one compound according to claim 1 and at least one further therapeutic agent related from the group consisting of interferon (IFN), interferon α-2a, interferon α-2b, consensus interferon (CIFN), ribavirin, amantadine, rimantadine, interleukine-12, ursodeoxycholic acid (UDCA), and glycyrrhizin.

18. A method for treating a patient suffering from a RNA viral infection which comprises administering to the patient an effective amount of at least one compound according to claim 1 and at least one further therapeutic agent chosen from interferon (IFN), interferon α-2a, interferon α-2b, consensus interferon (CIFN), ribavirin, amantadine, rimantadine, interleukine-12, ursodeoxycholic acid (UDCA), and glycyrrhizin.

19. The method of claim 18 wherein the RNA viral infection comprises at least one member selected from the group consisting of Influenza, Hantaan Virus, Crimean Congo hemorrhagic fever virus, HCV, HBV, Coxsackie A, Coxsackie B, Echo, Rhino viral infection, small pox viral infection, Ebola viral infection, polio viral infection and West Nile viral infection.
